(19) United States
(12) Patent Application Publication
(10) Pub. No.: US 2008/0076812 A1
(43) Pub. Date: Mar. 27, 2008

(54) Formulations of sitaxsentan sodium

(76) Inventors: Jinling Chen, Houston, TX (US); Sandy Koppenol, Lake Forest Park, WA (US); Lian Rajewski, Lawrence, KS (US); Andrew Trammel, Olathe, KS (US); Kent Amsberry, Fishers, IN (US); Aaron Schoeneman, Lee's Summit, MO (US)

Correspondence Address: JONES DAY, 222 EAST 41ST ST, NEW YORK, NY, 10017, US

(21) Appl. No.: 11/717,496

(22) Filed: Mar. 12, 2007

Related U.S. Application Data
(60) Provisional application No. 60/781,880, filed on Mar. 13, 2006.

(51) Int. Cl. A61K 31/42; C07D 267/04

(52) U.S. Cl. 514/380; 548/246

(57) ABSTRACT

Provided herein are stable lyophilized and oral formulations of sitaxsentan sodium. In certain embodiments the lyophilized formulations provided herein have improved stability upon reconstitution. Also provided are methods of making and using the formulations.

This application claims priority to U.S. provisional application Ser. No. 60/781,880 filed Mar. 13, 2006, entitled “FORMULATIONS OF SITAXSENTAN SODIUM” to Chen et al. The disclosure of the above referenced application is incorporated by reference herein.

### FIELD

Provided herein are formulations of sitaxsentan sodium and methods for treating endothelin-mediated disorders using the same. In certain embodiments, provided herein are lyophilized formulations. In certain embodiments, the formulations are oral tablets. Also provided are methods of making and using the formulations.

### BACKGROUND

Sitaxsentan sodium modulates activity of the endothelin family of peptides and is useful for the treatment of endothelin-mediated disorders. Due to the nature of these disorders, formulations containing sitaxsentan sodium may require storage for an extended period of time. In case of lyophilized powders, stability of the reconstituted formulations is important. The previously known lyophilized formulations of sitaxsentan sodium are not stable upon reconstitution. Therefore, stable formulations of this compound are desired.

### SUMMARY

In one embodiment, provided herein are lyophilized formulations of sitaxsentan sodium and methods for treatment of endothelin mediated disorders using the same. The formulations contain one or more antioxidants to prevent oxidation of sitaxsentan sodium. In one embodiment, the antioxidant is monothioglycerol, ascorbic acid, sodium bisulfite or sodium sulfite or a combination thereof. The formulations optionally further contain a buffer and/or a bulking agent, selected from sugars, polyalcohols, amino acids, polymers and polysaccharides.

In one embodiment, provided herein are oral tablet formulations of sitaxsentan sodium and methods for treatment of endothelin mediated disorders using the same. The tablets contain one or more excipients selected from a buffer, an antioxidant, a binding agent, a diluent, a lubricant and a coating agent.

Also provided are methods of making the formulations. Further provided are articles of manufacture containing packaging material, the stable formulation of sitaxsentan sodium and a label that indicates that the formulation is for the treatment of an endothelin mediated disorder.

### BRIEF DESCRIPTION OF FIGURES

FIG. 1 demonstrates small scale lyophilization of sitaxsentan sodium formulation containing various antioxidant systems (Formulas 1 A to 4A described in the examples correspond to samples in the figure as follows: IVA=A; IIA=B; IA=C; IIIA=D).

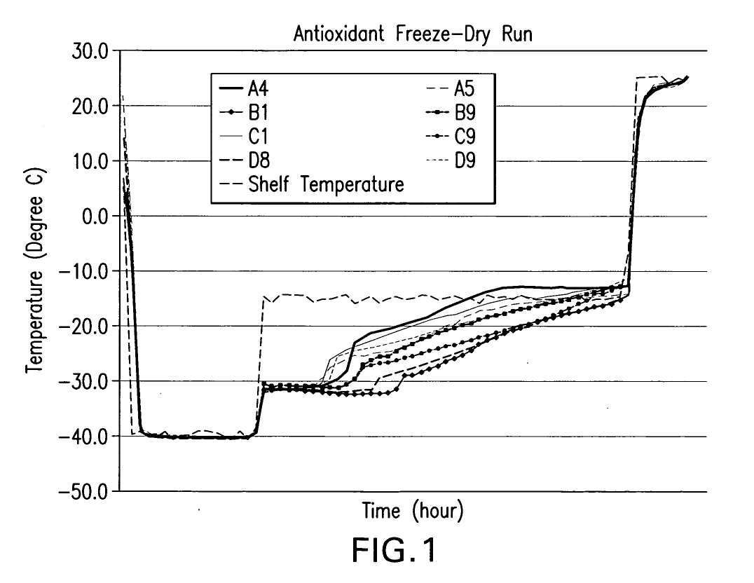

FIG. 2 demonstrates lyophilization of 25 mg/mL sitaxsentan sodium in 20 mM citrate buffer (pH 6), 4% dextrose with 2 mg/mL ascorbic acid, 6.6 mg/mL sodium bisulfite, and 2 mg/mL sodium sulfite for prototype stability.

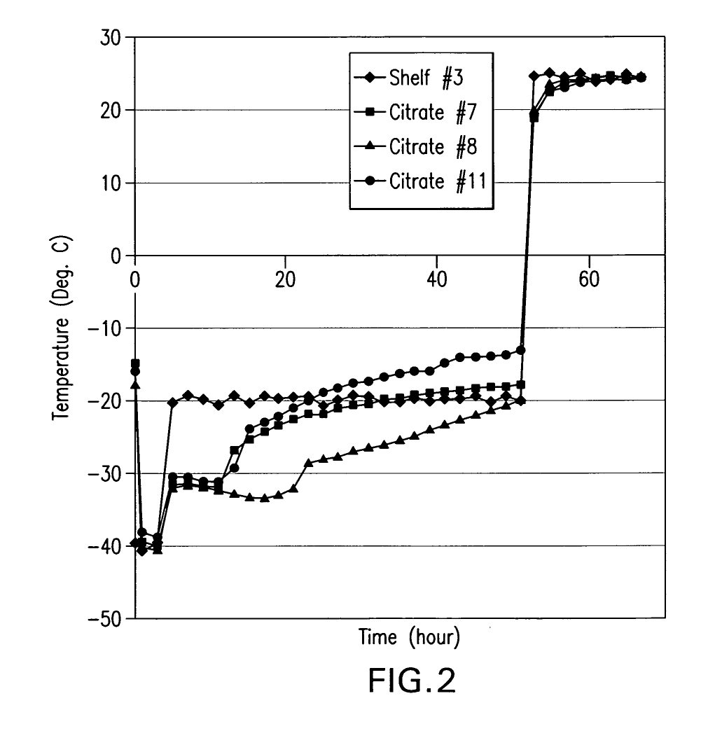

FIG. 3 demonstrates lyophilization of 25 mg/mL sitaxsentan sodium in 20 mM citrate buffer (pH 7) 4% dextrose with 10 mg/mL monothioglycerol.

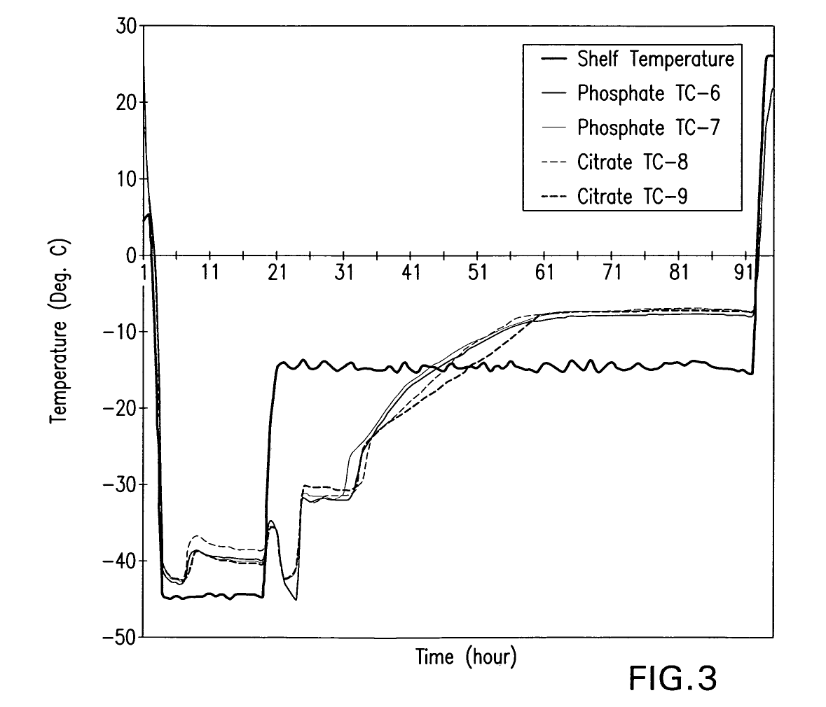

FIG. 4 demonstrates lyophilization of 25 mg/mL sitaxsentan sodium in 20 mM phosphate buffer (pH 7), 4% dextrose with 10 mg/mL monothioglycerol for prototype stability.

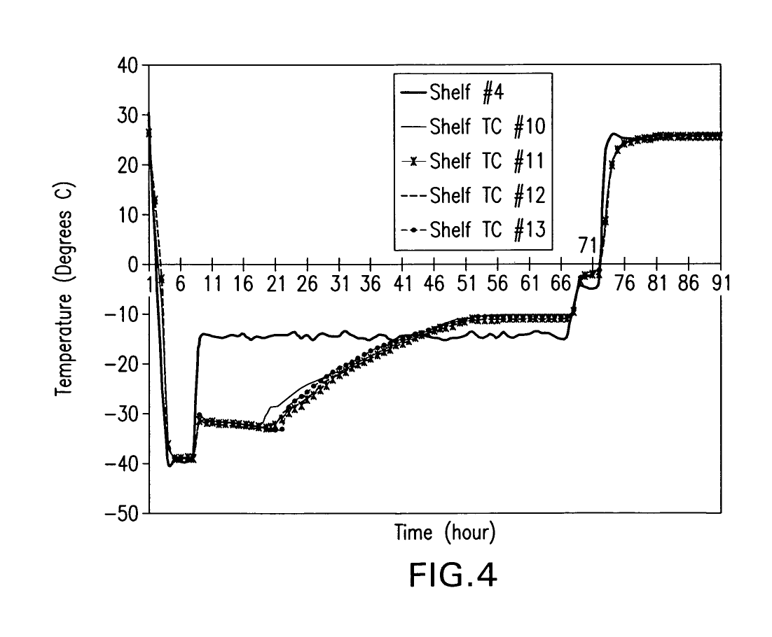

FIG. 5 illustrates lyophilization conditions for formulations 8a, 8b and 8c.

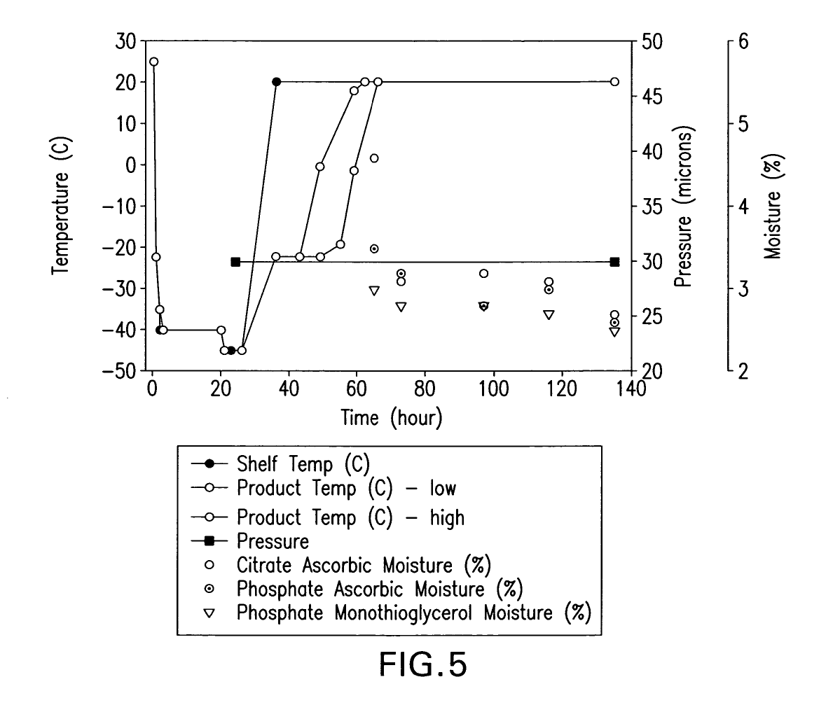

### DETAILED DESCRIPTION

A. Definitions

Unless defined otherwise, all technical and scientific terms used herein have the same meaning as is commonly understood by one of ordinary skill in the art. All patents, applications, published applications and other publications are incorporated by reference in their entirety. In the event that there are a plurality of definitions for a term herein, those in this section prevail unless stated otherwise.

As used herein “sitaxsentan” refers to N-(4-chloro-3-methyl-5-isoxazolyl)-2-[2-methyl-4,5-(methylenedioxy)phenylacetyl]-thiophene-3-sulfonamide. Sitaxsentan is also known as TBC 11251. Other chemical names for sitaxsentan include 4-chloro-3-methyl-5-(2-(2-(6-methylbenzo[d][1,3]dioxol-5-yl)acetyl)-3-thienylsulfonamido)isoxazole and N-(4-chloro-3-methyl-5-isoxazolyl)-2-[3,4-(methylenedioxy)-6-methylphenylacetyl]-thiophene-3-sulfonamide. The chemical structures of sitaxsentan and sitaxsentan sodium salt are described elsewhere herein.

As used herein “subject” is an animal, such as a mammal, including human, such as a patient.

As used herein, “an endothelin-mediated disorder” is a condition that is caused by abnormal endothelin activity or one in which compounds that inhibit endothelin activity have therapeutic use. Such disorders include, but are not limited to hypertension, cardiovascular disease, asthma, inflammatory diseases, opthalmologic disease, menstrual disorders, obstetric conditions, gastroenteric disease, renal failure, pulmonary hypertension, endotoxin shock, anaphylactic shock, or hemorrhagic shock.

As used herein, and unless otherwise specified, the terms “treat,” “treating” and “treatment” contemplate an action that occurs while a patient is suffering from the specified disease or disorder, which reduces the severity of the disease or disorder, or retards or slows the progression of the disease or disorder. Treatment also encompasses any pharmaceutical use of the compositions herein, such as use for treating pulmonary hypertension.

As used herein, amelioration of the symptoms of a particular disorder by administration of a particular pharmaceutical composition refers to any lessening, whether permanent or temporary, lasting or transient that can be attributed to or associated with administration of the composition.

As used herein, unless otherwise specified, the terms “prevent,” “preventing” and “prevention” contemplate an action that occurs before a patient begins to suffer from the specified disease or disorder, which inhibits or reduces the severity of the disease or disorder.

As used herein, and unless otherwise indicated, the terms “manage,” “managing” and “management” encompass preventing the recurrence of the specified disease or disorder in a patient who has already suffered from the disease or disorder, and/or lengthening the time that a patient who has suffered from the disease or disorder remains in remission. The terms encompass modulating the threshold, development and/or duration of the disease or disorder, or changing the way that a patient responds to the disease or disorder.

As used herein, and unless otherwise specified, the terms “therapeutically effective amount” and “effective amount” of a compound mean an amount sufficient to provide a therapeutic benefit in the treatment, prevent and/or management of a disease, to delay or minimize one or more symptoms associated with the disease or disorder to be treated. The terms “therapeutically effective amount” and “effective amount” can encompass an amount that improves overall therapy, reduces or avoids symptoms or causes of disease or disorder, or enhances the therapeutic efficacy of another therapeutic agent.

As used herein, and unless otherwise specified, the term “prophylactically effective amount” of a compound means an amount sufficient to prevent a disease or disorder, or one or more symptoms associated with the disease or disorder, or prevent its recurrence. The term “prophylactically effective amount” can encompass an amount that improves overall prophylaxis or enhances the prophylactic efficacy of another prophylactic agent.

The terms “co-administration” and “in combination with” include the administration of two therapeutic agents either simultaneously, concurrently or sequentially with no specific time limits. In one embodiment, both agents are present in the cell or in the patient's body at the same time or exert their biological or therapeutic effect at the same time. In one embodiment, the two therapeutic agents are in the same composition or unit dosage form. In another embodiment, the two therapeutic agents are in separate compositions or unit dosage forms. In some embodiments, a first agent can be administered prior to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of a second therapeutic agent.

B. Sitaxsentan Sodium

The chemical name for sitaxsentan is N-(4-chloro-3-methyl-5-isoxazolyl)-2-[2-methyl-4,5-(methylenedioxy)phenylacetyl]-thiophene-3-sulfonamide, and its structural formula is as follows:

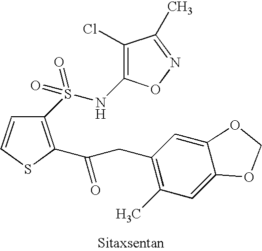

Sitaxsentan sodium has the formula:

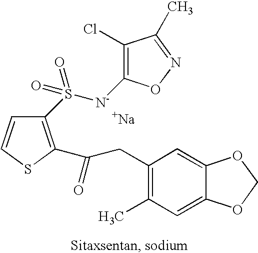

Sitaxsentan sodium is a potent endothelin receptor antagonist that has oral bioavailability in several species, a long duration of action, and high specificity for ETA receptors.

C. Exemplary Formulations

Provided herein are lyophilized and oral tablet formulations of sitaxsentan sodium.

Lyophilized Formulations

In certain embodiments, provided herein are lyophilized powder formulations of sitaxsentan sodium. In one embodiment, the lyophilized powder contains an antioxidant, a buffer and a bulking agent. In the lyophilized powders provided herein, the amount of sitaxsentan sodium present is in a range from about 25% to about 60% by total weight of the lyophilized powder. In certain embodiments, the amount of sitaxsentan sodium is from about 30% to about 50% or about 35% to about 45% by total weight of the lyophilized powder. In certain embodiments, the amount of sitaxsentan sodium is about 30%, 33%, 35%, 37%, 40%, 41%, 43%, 45%, 47%, 50%, 53%, 55% or 60% by total weight of the lyophilized powder. In one embodiment, the amount of sitaxsentan sodium in the lyophilized powder is about 41% by total weight of the lyophilized powder.

In certain embodiments, the lyophilized powder contains an antioxidant, such as sodium sulfite, sodium bisulfite, sodium metasulfite, monothioglycerol, ascorbic acid or a combination thereof. In one embodiment, the antioxidant is monothioglycerol. In one embodiment, the antioxidant is a combination of ascorbic acid, sodium sulfite and sodium bisulfite. In certain embodiments, the lyophilized formulations provided herein have improved stability upon reconstitution as compared to the known lyophilized formulations of sitaxsentan sodium (see WO 98/49162).

In certain embodiments, the antioxidant is monothioglycerol. In certain embodiments, the monothioglycerol is present in an amount ranging from about 10% to about 30% by total weight of the lyophilized powder. In certain embodiments, the monothioglycerol is present in an amount ranging from about 12% to about 25% or about 15% to about 20% by total weight of the lyophilized powder. In certain embodiments, the amount of monothioglycerol in the lyophilized powder is about 10%, 12%, 14%, 15%, 15.5%, 16%, 16.2%, 16.4%, 16.8%, 17%, 17.5%, 19%, 22%, 25% or 30% by total weight of the lyophilized powder. In certain embodiments, the amount of monothioglycerol is about 16.4% by total weight of the lyophilized powder.

In certain embodiments, the sodium sulfite is present in an amount from about 1% to about 6% by total weight of the lyophilized powder. In other embodiments, the sodium sulfite is present in an amount from about 1.5% to about 5% or about 2% to about 4%. In certain embodiments, the amount of sodium sulfite is about 1%, 1.5%, 2%, 2.5%, 3%, 3.3%, 3.5%, 3.8%, 4%, 4.5% or 5% by total weight of the lyophilized powder. In one embodiment, the amount of sodium sulfite is about 3.3% by total weight of the lyophilized powder.

In certain embodiments, the ascorbic acid is present in an amount from about 1% to about 6% by total weight of the lyophilized powder. In other embodiments, the ascorbic acid is present in an amount from about 1.5% to about 5% or about 2% to about 4%. In certain embodiments, the amount of ascorbic acid is about 1%, 1.5%, 2%, 2.5%, 3%, 3.3%, 3.5%, 3.8%, 4%, 4.5% or 5% by total weight of the lyophilized powder. In one embodiment, the amount of ascorbic acid is about 3.3% by total weight of the lyophilized powder.

In certain embodiments, the sodium bisulfite is present in an amount from about 5% to about 15% or about 8% to about 12% by total weight of the lyophilized powder. In certain embodiments, the sodium bisulfite is present in an amount from about 5%, 6%, 7%, 8%, 9%, 10%, 10.3%, 10.5%, 10.8%, 11%, 11.5%, 12% or 15% by total weight of the lyophilized powder. In one embodiment, the amount of sodium bisulfite is about 10.8% by total weight of the lyophilized powder.

In one embodiment, the antioxidant is a combination of ascorbic acid, sodium sulfite and sodium bisulfite. In one embodiment, the amount of ascorbic acid in the lyophilized powder is about 3.3%, the amount of sodium sulfite is about 3.3% and the amount of sodium bisulfite is about 10.8% by total weight of the lyophilized powder.

In one embodiment, the lyophilized powder also contains one or more of the following excipients: a buffer, such as sodium or potassium phosphate, or citrate buffer; and a bulking agent, such as glucose, dextrose, maltose, sucrose, lactose, sorbitol, mannitol, glycine, polyvinylpyrrolidone or dextran. In one embodiment, the bulking agent is selected from dextrose, D-mannitol and sorbitol.

In certain embodiments, the lyophilized powders provided herein contain a phosphate buffer. In certain embodiments, the phosphate buffer is present in a concentration of about 10 mM, about 15 mM, about 20 mM, about 25 mM or about 30 mM. In certain embodiments, the phosphate buffer is present in a concentration of 20 mM. In certain embodiments, the phosphate buffer is present in a concentration of 20 mM, and the constituted formulation has a pH of about 7.

In certain embodiments, the lyophilized powders provided herein contain a citrate buffer. In one embodiment, the citrate buffer is sodium citrate dihydrate. In certain embodiments, the amount of sodium citrate dihydrate is from about 5% to about 15%, about 6% to about 12% or about 7% to about 10% by total weight of the lyophilized powder. In certain embodiments, the amount of sodium citrate dihydrate in the lyophilized powder is about 5%, 6%, 7%, 7.5%, 8%, 8.3%, 8.5%, 8.8%, 9%, 9.5%, 10%, 12% or about 15% by total weight of the lyophilized powder. In certain embodiments, the constituted formulation has a pH of about 5 to 10, or about 6.

In certain embodiments, the lyophilized powder provided herein contains dextrose in an amount ranging from about 30% to about 60% by total weight of the lyophilized powder. In certain embodiments, the amount of dextrose is about 30%, 35%, 40%, 45%, 50% or 60% by total weight of the lyophilized powder. In certain embodiments, the amount of dextrose is about 40% by total weight of the lyophilized powder. In certain embodiments, the lyophilized powder provided herein contains mannitol in an amount ranging from about 20% to about 50% by total weight of the lyophilized powder. In certain embodiments, the amount of mannitol is about 20%, 25%, 30%, 32%, 32.5%, 32.8%, 33%, 34%, 37%, 40%, 45% or 50% by total weight of the lyophilized powder. In certain embodiments, the amount of mannitol is about 32.8% by total weight of the lyophilized powder.

In certain embodiments, the lyophilized powder provided herein contains about 41% of sitaxsentan sodium, about 3.3% ascorbic acid, about 3.3% sodium sulfite and about 10.8% mg sodium bisulfite, about 8.8% sodium citrate dihydrate and about 32.8% D-mannitol by total weight of the lyophilized powder. In certain embodiments, the lyophilized powder has the following composition:

Sitaxsentan Sodium Lyophilized Formulation

| Component | Quantity in a 10 mL vial (mg/vial) |
|---|---|
| Sitaxsentan Sodium | 250.0 |
| Sodium Citrate Dihydrate | 53.5 |
| L-Ascorbic Acid | 20.0 |
| D-Mannitol | 200.0 |
| Sodium Bisulfite | 66.0 |
| Sodium Sulfite | 20.0 |
| Sodium Hydroxide or | QS to pH 6 |
| Hydrochloride Acid | |

In certain embodiments, the lyophilized powder provided herein contains about 40 to about 30% of sitaxsentan sodium, about 4 to about 6% ascorbic acid, about 6 to about 8% sodium citrate dihydrate, about 50 to about 60% D-mannitol and about 1 to about 2% citric acid monohydrate by total weight of the lyophilized powder. In certain embodiments, the lyophilized powder provided herein contains about 33% of sitaxsentan sodium, about 5.3% ascorbic acid, about 7.6% sodium citrate dihydrate, about 53% D-mannitol and 0.13% citric acid monohydrate by total weight of the lyophilized powder. In one embodiment, the lyophilized powder has the following composition:

Sitaxsentan Sodium Lyophilized Formulation

| Component | Quantity in a 10 mL vial (mg/vial) |
|---|---|
| Sitaxsentan Sodium | 250.0 |
| Sodium Citrate Dihydrate | 57.1 |
| L-Ascorbic Acid | 40.0 |
| D-Mannitol | 400.0 |
| Citric Acid Monohydrate | 1.3 |
| Sodium Hydroxide or | QS to pH 6.8 |
| Hydrochloride Acid | |

In certain embodiments, the lyophilized powder provided herein contains about 40 to about 30% of sitaxsentan sodium, about 4 to about 6% ascorbic acid, about 3 to about 4% sodium phosphate dibasic heptahydrate, about 50 to about 60% D-mannitol and about 1.5 to about 2.5% sodium phosphate monobasic monohydrate by total weight of the lyophilized powder. In certain embodiments, the lyophilized powder provided herein contains about 34% of sitaxsentan sodium, about 5.5% ascorbic acid, about 3.7% sodium phosphate dibasic heptahydrate, about 55% D-mannitol and 1.9% sodium phosphate monobasic monohydrate by total weight of the lyophilized powder. In one embodiment, the lyophilized powder has the following composition:

Sitaxsentan Sodium Lyophilized Formulation

| Component | Quantity in a 10 mL vial (mg/vial) |
|---|---|
| Sitaxsentan Sodium | 250.0 |
| Sodium Phosphate Dibasic | 26.8 |
| Heptahydrate | |
| L-Ascorbic Acid | 40.0 |
| D-Mannitol | 400.0 |
| Sodium Phosphate Monobasic | 13.9 |
| Monohydrate | |
| Sodium Hydroxide or | QS to pH 6.8 |
| Hydrochloride Acid | |

The lyophilized formulations of sitaxsentan sodium provided herein can be administered to a patient in need thereof using standard therapeutic methods for delivering sitaxsentan sodium including, but not limited to, the methods described herein. In one embodiment, the lyophilized sitaxsentan sodium is administered by dissolving a therapeutically effective amount of the lyophilized sitaxsentan sodium provided herein in a pharmaceutically acceptable solvent to produce a pharmaceutically acceptable solution, and administering the solution (such as by intravenous injection) to the patient.

The lyophilized sitaxsentan sodium formulation provided herein can be constituted for parenteral administration to a patient using any pharmaceutically acceptable diluent. Such diluents include, but are not limited to Sterile Water for Injection, USP, Sterile Bacteriostatic Water for Injection, saline, USP (benzyl alcohol or parabens preserved). Any quantity of diluent may be used to constitute the lyophilized sitaxsentan sodium formulation such that a suitable solution for injection is prepared. Accordingly, the quantity of the diluent must be sufficient to dissolve the lyophilized sitaxsentan sodium. In one embodiment, 10-50 mL or 10 to 20 mL of a diluent are used to constitute the lyophilized sitaxsentan sodium formulation to yield a final concentration of, about 1-50 mg/mL, about 5-40 mg/mL, about 10-30 mg/mL or 10-25 mg/mL. In certain embodiments, the final concentration of sitaxsentan sodium in the reconstituted solution is about 25 mg/mL or about 12.5 mg/mL. The precise amount depends upon the indication treated. Such amount can be empirically determined. In some embodiments, the pH of the reconstituted solution is about 5 to about 10 or about 6 to about 8. In some embodiments, the pH of the reconstituted solution is about 5, 6, 7, 8, 9 or 10.

Constituted solutions of lyophilized sitaxsentan sodium can be administered to a patient promptly upon constitution. Alternatively, constituted solutions can be stored and used within about 1-72 hours, about 1-48 hours or about 1-24 hours. In some embodiments, the solution is used within 1 hour of preparation.

Tablet Formulations

In certain embodiments, provided herein are oral tablets containing sitaxsentan sodium. In one embodiment, the oral tablet further contains a buffer. In one embodiment, the oral tablet further contains an antioxidant. In one embodiment, the oral tablet further contains a moisture barrier coating.

In some embodiments, the tablets contain excipients, including, but not limited to an antioxidant, such as sodium ascorbate, glycine, sodium metabisulfite, ascorbyl palmitate, disodium edetate (EDTA) or a combination thereof; a binding agent, such as hydroxypropyl methylcellulose; a diluent, such as lactose monohydrate, including lactose monohydrate fast flo (intragranular) and lactose monohydrate fast flo (extragranular) and microcrystalline cellulose and a buffer, such as phosphate buffer. The tablet can further contain one or more excipients selected from a lubricant, a disintegrant and a bulking agent.

In certain embodiments, the amount of sitaxsentan sodium in the oral tablet is from about 5% to about 40% of the total weight of the composition. In certain embodiments, the amount of sitaxsentan sodium is from about 7% to about 35%, 10% to about 30%, 12% to about 32%, 15% to about 30%, 17% to about 27%, 15% to about 25% of the total weight of the composition. In certain embodiments, the amount of sitaxsentan sodium is about 5%, 7%, 9%, 10%, 12%, 15%, 17%, 20%, 22%, 25%, 27%, 30%, 35% or 40% of the total weight of the composition. In certain embodiments, the amount of sitaxsentan sodium is about 20%.

In certain embodiments, the oral tablet contains about 10 mg, 20 mg, 25 mg, 30 mg, 40 mg, 50 mg, 60 mg, 70 mg, 80 mg, 90 mg, 100 mg, 125 mg, 150 mg, 175 mg, 200 mg, 225 mg, 250 mg, 275 mg, 280 mg, 300 mg or 350 mg of sitaxsentan sodium.

In certain embodiments, the tablets contain a combination of two antioxidants, such as ascorbyl palmitate and EDTA, disodium. In certain embodiments, the amount of ascorbyl palmitate in the formulation is in a range from about 0.05% to about 3% of the total weight of the tablet. In other embodiments, the amount of ascorbyl palmitate is in a range from about 0.07% to about 1.5%, 0.1% to about 1% or 0.15% to about 0.5% of the total weight of the tablet. In certain embodiments, the amount of ascorbyl palmitate in the formulation is about 0.05%, 0.07%, 0.09%, 0.1%, 0.12%, 0.15%, 0.17%, 0.18%, 0.2%, 0.23%, 0.25%, 0.27%, 0.3%, 0.35%, 0.4%, 0.45%, 0.5%, 0.7% or 1%. In certain embodiments, the amount of ascorbyl palmitate in the formulation is about 0.2% of the total weight of the tablet.

In certain embodiments, the amount of ascorbyl palmitate in the oral tablet is from about 0.1 mg to about 5 mg, about 0.5 mg to about 4 mg, about 0.7 mg to about 3 mg or about 1 mg to about 2 mg. In certain embodiments, the amount of ascorbyl palmitate in the oral tablet is about 0.1 mg, 0.5 mg, 0.7 mg, 1 mg, 1.3 mg, 1.5 mg, 1.7 mg, 2 mg, 2.5 mg or about 3 mg. In certain embodiments, the amount of ascorbyl palmitate in the formulation is about 1 mg.

In certain embodiments, the amount of EDTA, disodium in the formulation is in a range from about 0.05% to about 3% by weight of the total weight of the tablet. In other embodiments, the amount of EDTA, disodium is in a range from about 0.07% to about 1.5%, 0.1% to about 1% or 0.15% to about 0.5% of the total weight of the tablet. In certain embodiments, the amount of EDTA, disodium in the formulation is about 0.05%, 0.07%, 0.09%, 0.1%, 0.12%, 0.15%, 0.17%, 0.18%, 0.2%, 0.23%, 0.25%, 0.27%, 0.3%, 0.35%, 0.4%, 0.45%, 0.5%, 0.7% or 1%. In certain embodiments, the amount of EDTA, disodium in the formulation is about 0.2% of the total weight of the tablet.

In certain embodiments, the amount of EDTA, disodium in the oral tablet is from about 0.1 mg to about 5 mg, about 0.5 mg to about 4 mg, about 0.7 mg to about 3 mg or about 1 mg to about 2 mg. In certain embodiments, the amount of EDTA, disodium in the oral tablet is about 0.1 mg, 0.5 mg, 0.7 mg, 1 mg, 1.3 mg, 1.5 mg, 1.7 mg, 2 mg, 2.5 mg or about 3 mg. In certain embodiments, the amount of EDTA, disodium in the oral tablet is about 1 mg.

In certain embodiments, the tablets contain a combination of diluents, such as microcrystalline cellulose (AVICEL PH 102), lactose monohydrate fast flo (intragranular) and lactose monohydrate fast flo (extragranular). In certain embodiments, the amount of lactose monohydrate fast flo (intragranular) in the oral tablet is from about 5% to about 30% of the total weight of the composition. In certain embodiments, the amount of lactose monohydrate fast flo (intragranular) is from about 7% to about 25%, from about 10% to about 20% or from about 13% to about 20% of the total weight of the tablet. In certain embodiments, the amount of lactose monohydrate fast flo (intragranular) is about 5%, 7%, 10%, 13%, 14%, 15%, 15.5%, 16%, 16.1%, 16.2%, 16.3%, 16.4%, 16.5%, 16.6%, 16.7%, 16.8%, 16.9%, 17%, 17.5%, 18%, 18.5%, 19%, 20%, 25% or 30% of the total weight of the tablet. In certain embodiments, the amount of lactose monohydrate fast flo (intragranular) is about 16.9% of the total weight of the tablet.

In certain embodiments, the amount of lactose monohydrate fast flo (intragranular) is from about 40 mg to about 100 mg, from about 45 mg to about 95 mg or from about 50 mg to about 90 mg. In certain embodiments, the amount of lactose monohydrate fast flo (intragranular) is about 40 mg, 45 mg, 50 mg, 55 mg, 60 mg, 65 mg, 70 mg, 75 mg, 80 mg, 81 mg, 82 mg, 83 mg, 83.5 mg, 84 mg, 84.1 mg, 84.2 mg, 84.3 mg, 84.4 mg, 84.5 mg, 84.6 mg, 84.7 mg, 85 mg, 85.5 mg, 90 mg, 90.5 mg or 100 mg. In certain embodiments, the amount of lactose monohydrate fast flo (intragranular) is about 84.3 mg.

In certain embodiments, the amount of lactose monohydrate fast flo (extragranular) is from about 7% to about 25%, from about 10% to about 20% or from about 13% to about 20% of the total weight of the tablet. In certain embodiments, the amount of lactose monohydrate fast flo (extragranular) is about 5%, 7%, 10%, 13%, 14%, 15%, 15.5%, 16%, 16.1%, 16.2%, 16.3%, 16.4%, 16.5%, 16.6%, 16.7%, 16.8%, 16.9%, 17%, 17.5%, 18%, 18.5%, 19%, 20%, 25% or 30% of the total weight of the tablet. In certain embodiments, the amount of lactose monohydrate fast flo (extragranular) is about 16.4% of the total weight of the tablet. In certain embodiments, the amount of lactose monohydrate fast flo (extragranular) in the oral tablet is from about 40 mg to about 100 mg, from about 45 mg to about 95 mg or from about 50 mg to about 90 mg. In certain embodiments, the amount of lactose monohydrate fast flo (extragranular) is about 40 mg, 45 mg, 50 mg, 55 mg, 60 mg, 65 mg, 70 mg, 75 mg, 80 mg, 81 mg, 81.3 mg, 81.5 mg, 81.8 mg, 82 mg, 82.3 mg, 82.5 mg, 82.7 mg, 83 mg, 83.5 mg, 84 mg, 85 mg, 85.5 mg, 90 mg, 90.5 mg or 100 mg. In certain embodiments, the amount of lactose monohydrate fast flo (intragranular) is about 82 mg.

In certain embodiments, the amount of microcrystalline cellulose (Avicel PH 102) in the oral tablet is from about 10% to about 50% of the total weight of the composition. In certain embodiments, the amount of microcrystalline cellulose (Avicel PH 102) is from about 15% to about 45%, from about 20% to about 43% or from about 25% to about 40% of the total weight of the tablet. In certain embodiments, the amount of microcrystalline cellulose (Avicel PH 102) is about 15%, 17%, 20%, 23%, 25%, 27%, 30%, 32%, 34%, 35%, 37%, 40%, 42%, 45% or 50% of the total weight of the tablet. In certain embodiments, the amount of microcrystalline cellulose (Avicel PH 102) is about 35% of the total weight of the tablet.

In certain embodiments, the amount of microcrystalline cellulose (Avicel PH 102) in the oral tablet is from about 130 mg to about 300 mg. In certain embodiments, the amount of microcrystalline cellulose (Avicel PH 102) is from about 140 mg to about 275 mg or about 150 mg to about 250 mg. In certain embodiments, the amount of microcrystalline cellulose (Avicel PH 102) is about 150 mg, 160 mg, 165 mg, 170 mg, 175 mg, 180 mg, 185 mg, 190 mg or 200 mg. In certain embodiments, the amount of microcrystalline cellulose (Avicel PH 102) in the oral tablet is about 175 mg.

In certain embodiments, the binding agent is hydroxypropyl methylcellulose (E-5P). In certain embodiments, the amount of hydroxypropyl methylcellulose (E-5P) in the tablet is from about 0.5% to about 20% of the total weight of the composition. In certain embodiments, the amount of hydroxypropyl methylcellulose (E-5P) is from about 1% to about 15%, from about 2% to about 10% or from about 3% to about 8% of the total weight of the tablet. In certain embodiments, the amount of hydroxypropyl methylcellulose (E-5P) is about 1%, 2%, 3%, 4%, 5%, 6%, 7%, 8%, 9% or 10% of the total weight of the tablet. In certain embodiments, the amount of hydroxypropyl methylcellulose (E-5P) is about 5% of the total weight of the tablet.

In certain embodiments, the amount of hydroxypropyl methylcellulose (E-5P) in the tablet is from about 5 mg to about 50 mg, about 10 mg to about 40 mg or about 15 mg to about 30 mg. In certain embodiments, the amount of hydroxypropyl methylcellulose (E-5P) in the tablet is about 10 mg, 15 mg, 20 mg, 22 mg, 25 mg, 27 mg, 30 mg, 35 mg or about 40 mg. In certain embodiments, the amount of hydroxypropyl methylcellulose (E-5P) in the tablet is about 25 mg.

The formulations of sitaxsentan sodium provided herein are stable at neutral pH. In certain embodiments, buffer agent mixture, such as sodium phosphate monobasic monohydrate and sodium phosphate dibasic anhydrous is used to improve drug stability in the tablets. In certain embodiments, the amount of sodium phosphate, monobasic monohydrate ranges from about 0.05% to about 3% by weight of the total weight of the tablet. In other embodiments, the amount of sodium phosphate, monobasic monohydrate is in a range from about 0.07% to about 1.5%, 0.1% to about 1% or 0.15% to about 0.5% of the total weight of the tablet. In certain embodiments, the amount of sodium phosphate, monobasic monohydrate in the formulation is about 0.05%, 0.07%, 0.09%, 0.1%, 0.12%, 0.15%, 0.17%, 0.18%, 0.2%, 0.23%, 0.25%, 0.27%, 0.3%, 0.35%, 0.4%, 0.45%, 0.5%, 0.7% or 1.% of the total weight of the tablet. In certain embodiments, the amount of sodium phosphate, monobasic monohydrate in the formulation is about 0.1% of the total weight of the tablet.

In certain embodiments, the amount of sodium phosphate, monobasic monohydrate in the oral tablet is from about 0.1 mg to about 3 mg, about 0.2 mg to about 2.5 mg, about 0.5 mg to about 2 mg or about 0.6 mg to about 1 mg. In certain embodiments, the amount of sodium phosphate, monobasic monohydrate in the oral tablet is about 0.1 mg, 0.2 mg, 0.3 mg, 0.4 mg, 0.5 mg, 0.6 mg, 0.7 mg, 0.8 mg, 0.9 mg or about 1 mg. In certain embodiments, the amount of sodium phosphate, monobasic monohydrate in the oral tablet is about 0.6 mg.

In certain embodiments, the amount of sodium phosphate, dibasic anhydrous ranges from about 0.05% to about 3% by weight of the total weight of the tablet. In other embodiments, the amount of sodium phosphate dibasic is in a range from about 0.07% to about 1.5%, 0.1% to about 1% or 0.15% to about 0.5% of the total weight of the tablet. In certain embodiments, the amount of sodium phosphate dibasic in the formulation is about 0.05%, 0.07%, 0.09%, 0.1%, 0.12%, 0.15%, 0.17%, 0.18%, 0.2%, 0.23%, 0.25%, 0.27%, 0.3%, 0.35%, 0.4%, 0.45%, 0.5%, 0.7% or 1.% of the total weight of the tablet. In certain embodiments, the amount of sodium phosphate dibasic in the formulation is about 0.2% of the total weight of the tablet.

In certain embodiments, the amount of sodium phosphate, dibasic anhydrous in the oral tablet is from about 0.1 mg to about 3.5 mg, about 0.5 mg to about 2.5 mg, or about 0.7 mg to about 2 mg. In certain embodiments, the amount of sodium phosphate, dibasic anhydrous in the oral tablet is about 0.1 mg, 0.3 mg, 0.5 mg, 0.7 mg, 0.9 mg, 1 mg, 1.1 mg, 1.3 mg, 1.5 mg, 1.7 mg or 2 mg. In certain embodiments, the amount of sodium phosphate, dibasic anhydrous in the oral tablet is about 1.1 mg.

In certain embodiments, the tablet contains disintegrants, such as sodium starch glycoloate (intragranular) and sodium starch glycoloate (extragranular). In certain embodiments, the amount of sodium starch glycoloate (intragranular) in the tablet is from about 0.1% to about 10% of the total weight of the composition. In certain embodiments, the amount of sodium starch glycoloate (intragranular) is from about 0.5% to about 8%, from about 1% to about 5% or from about 2% to about 4% of the total weight of the tablet. In certain embodiments, the amount of sodium starch glycoloate (intragranular) is about 0.5%, 1%, 1.5%, 1.7%, 2%, 2.3%, 2.5%, 2.7%, 3%, 3.5%, 4% or 5% of the total weight of the tablet. In certain embodiments, the amount of Sodium Starch Glycoloate (intragranular) is about 2.5% of the total weight of the tablet. In certain embodiments, the amount of sodium starch glycoloate (intragranular) is from about 30 mg to about 5 mg, from about 20 mg to about 10 mg, from about 15 to about 10 mg. In certain embodiments, the amount of sodium starch glycoloate (intragranular) is about 5 mg, 7 mg, 10 mg, 11 mg, 11.5 mg, 12 mg, 12.5 mg, 13 mg, 15 mg or 20 mg. In certain embodiments, the amount of sodium starch glycoloate (intragranular) is about 12.5 mg.

In certain embodiments, the amount of sodium starch glycoloate (extragranular) in the tablet is from about 0.1% to about 10% of the total weight of the composition. In certain embodiments, the amount of sodium starch glycoloate (extragranular) is from about 0.5% to about 8%, from about 1% to about 5% or from about 2% to about 4% of the total weight of the tablet. In certain embodiments, the amount of sodium starch glycoloate (extragranular) is about 0.5%, 1%, 1.5%, 1.7%, 2%, 2.3%, 2.5%, 2.7%, 3%, 3.5%, 4% or 5% of the total weight of the tablet. In certain embodiments, the amount of sodium starch glycoloate (extragranular) is about 2.5% of the total weight of the tablet. In certain embodiments, the amount of sodium starch glycoloate (extragranular) is from about 30 mg to about 5 mg, from about 20 mg to about 10 mg or from about 15 to about 10 mg. In certain embodiments, the amount of sodium starch glycoloate (extragranular) is about 5 mg, 7 mg, 10 mg, 11 mg, 11.5 mg, 12 mg, 12.5 mg, 13 mg, 15 mg or 20 mg. In certain embodiments, the amount of sodium starch glycoloate (extragranular) is about 12.5 mg.

In certain embodiments, the tablet contains a lubricant, such as magnesium stearate. In certain embodiments, the amount of magnesium stearate in the tablet is from about 0.1% to about 8% of the total weight of the composition. In certain embodiments, the amount of magnesium stearate is from about 0.5% to about 6%, from about 0.7% to about 5% or from about 1% to about 4% of the total weight of the tablet. In certain embodiments, the amount of magnesium stearate is about 0.5%, 0.7%, 1%, 1.2%, 1.5%, 1.7%, 2%, 2.5% or 3% of the total weight of the tablet. In certain embodiments, the amount of magnesium stearate is about 2.5% of the total weight of the tablet. In certain embodiments, the amount of magnesium stearate in the tablet is from about 15 mg to about 1 mg. In certain embodiments, the amount of magnesium stearate is from about 10 mg to about 3 mg or from about 7 mg to about 5 mg. In certain embodiments, the amount of magnesium stearate is about 3 mg, 4 mg, 4.5 mg, 5 mg, 6 mg, 7 mg, 8 mg, 9 mg or 10 mg. In certain embodiments, the amount of magnesium stearate is about 5 mg.

In one embodiment, the tablet formulations provided herein contain a moisture barrier coating. Suitable coating materials are known in the art and include, but are not limited to coating agents either of cellulose origin such as hydroxypropylmethylcellulose (Sepifilm®, Pharmacoat), or of polyvinyl origin of Sepifilm® ECL type, or of saccharose origin such as the sugar for sugar-coating of Sepisperse DR, AS, AP OR K (coloured) type, such as Sepisperse Dry 3202 Yellow, Blue Opadry, Eudragit EPO and Opadry AMB. Without being bound by any particular theory, it is believed that the coating serves as a moisture barrier to hinder oxidation of sitaxsentan sodium. In certain embodiments, the coating materials are Sepifilm® LP014/Sepisperse Dry 3202 Yellow (Sepifilm® Sepisperse) (3/2 wt/wt) at from about 1 to about 7% or about 4% tablet weight gain. In certain embodiments, the coating material is Sepifilm® LP014/Sepisperse Dry 3202 Yellow (Sepifilm®/Sepisperse). In certain embodiments, the Sepifilm®/Sepisperse ratio is 1:2, 1:1 or 3:2 wt/wt. In certain embodiments, the Sepifilm®/Sepisperse coating is at about 1%, 2%, 3%, 4%, 5%, 6% or 7% tablet weight gain. In certain embodiments, the Sepifilm®/Sepisperse coating is at about 1.6% tablet weight gain. In certain embodiments, the Sepisperse Dry 3202 (yellow) is at about 0.5%, 0.8%, 1%, 1.3%, 1.6%, 2%, 2.4%, 2.5%, 3% or 4% tablet weight gain. In certain embodiments, the Sepisperse Dry 3202 (yellow) is at about 2.4% tablet weight gain. In certain embodiments, the Sepisperse Dry 3202 (yellow) is at about 1 mg, 3 mg, 5 mg, 6 mg, 7 mg, 8 mg, 9 mg, 10 mg, 13 mg 15 mg or 20 mg per tablet. In certain embodiments, the Sepisperse Dry 3202 (yellow) is at about 8 mg per tablet. In certain embodiments, the Sepifilm® LP 014 is at about 0.5%, 1%, 1.5%, 2%, 2.2%, 2.4%, 2.6%, 3%, 3.5% or 4% tablet weight gain. In certain embodiments, the Sepifilm® LP 014 is at about 2.4% tablet weight gain. In certain embodiments, the Sepifilm® LP 014 is at about 5 mg, 7 mg, 9 mg, 10 mg, 11 mg, 12 mg, 13 mg, 15 mg, 17 mg or 20 mg per tablet. In certain embodiments, the Sepifilm®(t LP 014 coating is at about 12 mg per tablet.

In certain embodiments, the tablet contains sitaxsentan sodium, microcrystalline cellulose, lactose monohydrate fast flo (intragranular), lactose monohydrate fast flo (extragranular), hydroxypropyl methylcellulose E-5P, ascorbyl palmitate, disodium EDTA, sodium phosphate monobasic, monohydrate, sodium phosphate dibasic, anhydrous, Sodium Starch Glycoloate (intragranular), Sodium Starch Glycoloate (extragranular), magnesium stearate and a coating of Sepifilm® LP014/Sepisperse Dry 3202 Yellow.

In certain embodiments, the tablet contains about 20% sitaxsentan sodium, about 35% microcrystalline cellulose, about 16.9% lactose monohydrate fast flo (intragranular), about 16.4% lactose monohydrate fast flo (extragranular), about 5.0% hydroxypropyl methylcellulose E-5P, about 0.2% ascorbyl palmitate, about 0.2% disodium (EDTA), about 0.1% sodium phosphate monobasic, monohydrate, about 0.2% sodium phosphate dibasic, anhydrous, about 2.5% Sodium Starch Glycoloate (extragranular), about 2.5% Sodium Starch Glycoloate (intragranular) and about 1% magnesium stearate. The tablet further contains a coating of Sepifilm® LP014 at about 2.4% weight gain and Sepisperse Dry 3202 Yellow at about 1.6% weight gain.

In certain embodiments, the oral tablet provided herein is a 500 mg tablet that contains about 100 mg sitaxsentan sodium, about 1.0 mg ascorbyl palmitate, about 1.0 mg disodium edetate (EDTA), about 25 mg hydroxypropyl methylcellulose E-5P, about 84.3 lactose monohydrate fast flo (intragranular), about 82 mg lactose monohydrate fast flo (extragranular), about 175 mg microcrystalline cellulose, about 0.6 mg sodium phosphate monobasic, monohydrate, about 1.1 mg sodium phosphate dibasic, anhydrous, about 12.5 mg Sodium Starch Glycoloate (extragranular), about 12.5 mg Sodium Starch Glycoloate (intragranular), about 5 mg magnesium stearate, non-bovine and about 192.5 mg purified water. The tablet further contains a coating of Sepifilm® LP014 at about 12 mg and Sepisperse Dry 3202 Yellow at about 8 mg.

D. Dosages

In human therapeutics, the physician will determine the dosage regimen that is most appropriate according to a preventive or curative treatment and according to the age, weight, stage of the disease and other factors specific to the subject to be treated. In certain embodiments, dose rates of sitaxsentan sodium are from about 1 to about 350 mg per day for an adult, from about 1 to about 300 mg per day, from about 5 to about 250 mg per day, from about 5 to about 250 mg per day or from about 10 to 50 mg per day for an adult. Dose rates of from about 50 to about 300 mg per day are also contemplated herein. In certain embodiments, doses are about 5 mg, 10 mg, 15 mg, 20 mg, 25 mg, 30 mg, 35 mg, 40 mg, 45 mg, 50 mg, 60 mg, 70 mg, 80 mg, 100 mg, 125 mg, 150 mg, 175 mg or 200 mg per day per adult.

The amount of sitaxsentan sodium in the formulations provided herein which will be effective in the prevention or treatment of a disorder or one or more symptoms thereof will vary with the nature and severity of the disease or condition, and the route by which the active ingredient is administered. The frequency and dosage will also vary according to factors specific for each subject depending on the specific therapy (e.g., therapeutic or prophylactic agents) administered, the severity of the disorder, disease, or condition, the route of administration, as well as age, body, weight, response, and the past medical history of the subject.

Exemplary doses of a formulation include milligram or microgram amounts of the active compound per kilogram of subject or sample weight (e.g., from about 1 micrograms per kilogram to about 3 milligrams per kilogram, from about 10 micrograms per kilogram to about 3 milligrams per kilogram, from about 100 micrograms per kilogram to about 3 milligrams per kilogram, or from about 100 microgram per kilogram to about 2 milligrams per kilogram). In certain embodiments, the amount of sitaxsentan sodium administered is from about 0.01 to about 3 mg/kg for a subject in need thereof. In certain embodiments, the amount of sitaxsentan sodium administered is about 0.01, 0.05, 0.1, 0.2, 0.4, 0.8, 1.5, 2 or 3 mg/kg of a subject. In the certain embodiments, the administration of sitaxsentan sodium is by intravenous injection.

It may be necessary to use dosages of the active ingredient outside the ranges disclosed herein in some cases, as will be apparent to those of ordinary skill in the art. Furthermore, it is noted that the clinician or treating physician will know how and when to interrupt, adjust, or terminate therapy in conjunction with subject response.

Different therapeutically effective amounts may be applicable for different diseases and conditions, as will be readily known by those of ordinary skill in the art. Similarly, amounts sufficient to prevent, manage, treat or ameliorate such disorders, but insufficient to cause, or sufficient to reduce, adverse effects associated with the composition provided herein are also encompassed by the above described dosage amounts and dose frequency schedules. Further, when a subject is administered multiple dosages of a composition provided herein, not all of the dosages need be the same. For example, the dosage administered to the subject may be increased to improve the prophylactic or therapeutic effect of the composition or it may be decreased to reduce one or more side effects that a particular subject is experiencing.

In another embodiment, the dosage of the formulation provided herein is administered to prevent, treat, manage, or ameliorate a disorder, or one or more symptoms thereof in a subject in a unit dose contain sitaxsentan sodium from about 1 mg to 300 mg, 50 mg to 250 mg or 75 mg to 200 mg.

In certain embodiments, administration of the same formulation provided herein may be repeated and the administrations may be separated by at least 1 day, 2 days, 3 days, 5 days, 10 days, 15 days, 30 days, 45 days, 2 months, 75 days, 3 months, or 6 months.

E. Methods of Preparation

Sitaxsentan sodium can be prepared by methods known in the art. An exemplary methods for the preparation are described in Example 1. (Also see, U.S. Pat. Nos. 5,783,705, 5,962,490 and 6,248,767; and Wu et al., J. Med. Chem. 1997, 40, 1690-1697).

The lyophilized and tablet formulations of sitaxsentan sodium can be prepared by methods known in the art and as described herein. In one embodiment, the process for making lyophilized formulation involves lyophilizing a solution of sitaxsentan sodium using a primary drying stage of duration from about 2 to 10 hours, or about 4 hours at from about −20° C. to about −60° C., or at about −40° C. The process further involves a secondary drying stage of duration for about 30 hours to about 70 hours, or about 50 hours at from about −30° C. to about −5° C. An exemplary process for producing the lyophilized formulations is described in Examples section.

F. Evaluation of the Activity

Standard physiological, pharmacological and biochemical procedures are available and are known to one of skill in the art (see, for example U.S. Pat. Nos. 6,432,994; 6,683,103; 6,686,382; 6,248,767; 6,852,745; 5,783,705; 5,962,490; 5,594,021; 5,571,821; 5,591,761; 5,514,691. 5,352,800, 5,334,598, 5,352,659, 5,248,807, 5,240,910, 5,198,548, 5,187,195, 5,082,838, 6,953,780, 6,946,481, 6,852,745, 6,835,741, 6,673,824, 6,670,367 and 6,670,362) to test the efficacy of sitaxsentan sodium formulations in the methods provided herein.

G. Methods of Treatment

Methods for the treatment of endothelin-mediated disorders by administering the lyophilized formulations provided herein. In certain embodiments, the disorder is selected from hypertension, cardiovascular disease, asthma, pulmonary hypertension, inflammatory diseases, opthalmologic disease, menstrual disorders, obstetric conditions, wounds, gastroenteric disease, renal failure, immunosuppressant-mediated renal vasoconstriction, erythropoietin-mediated vasoconstriction, endotoxin shock, anaphylactic shock and hemorrhagic shock. In one embodiment, the disorder is pulmonary hypertension.

H. Combination Therapy

Sitaxsentan sodium formulations provided herein can be employed alone or in combination with other suitable therapeutic agents useful in the treatment of the diseases treated by these formulations. For example, the formulations can be administered in combination with other compounds known to modulate the activity of endothelin receptor.

Further, the formulations provided herein can be employed in combination with endothelin antagonists known in the art and include, but are not limited to a fermentation product of Streptomyces misakiensis, designated BE-18257B which is a cyclic pentapeptide, cyclo(D-Glu-L-Ala-allo-D-lle-L-Leu-D-Trp); cyclic pentapeptides related to BE-18257B, such as cyclo(D-Asp-Pro-D-Val-Leu-D-Trp) (BQ-123) (see, U.S. Pat. No. 5,114,918 to Ishikawa et al.; see, also, EP A1 0 436 189 to BANYU PHARMACEUTICAL CO., LTD (Oct. 7, 1991)); and other peptide and non-peptidic ETA antagonists have been identified in, for example, U.S. Pat. Nos. 6,432,994; 6,683,103; 6,686,382; 6,248,767; 6,852,745; 5,783,705; 5,962,490; 5,594,021; 5,571,821; 5,591,761; 5,514,691; 5,352,800; 5,334,598; 5,352,659; 5,248,807; 5,240,910; 5,198,548; 5,187,195; 5,082,838; 6,953,780; 6,946,481; 6,852,745; 6,835,741; 6,673,824; 6,670,367; and 6,670,362. These include other cyclic pentapeptides, acyltripeptides, hexapeptide analogs, certain anthraquinone derivatives, indanecarboxylic acids, certain N-pyriminylbenzenesulfonamides, certain benzenesulfonamides, and certain naphthalenesulfonamides (Nakajima et al. (1991) J. Antibiot. 44:1348-1356; Miyata et al. (1992) J. Antibiot. 45:74-8; Ishikawa et al. (1992) J. Med. Chem. 35:2139-2142; U.S. Pat. No. 5,114,918 to Ishikawa et al.; EP A1 0 569 193; EP A1 0 558 258; EP A1 0 436 189 to BANYU PHARMACEUTICAL CO., LTD (Oct. 7, 1991); Canadian Patent Application 2,067,288; Canadian Patent Application 2,071,193; U.S. Pat. No. 5,208,243; U.S. Pat. No. 5,270,313; U.S. Pat. No. 5,612,359, U.S. Pat. No. 5,514,696, U.S. Pat. No. 5,378,715; Cody et al. (1993) Med. Chem. Res. 3:154-162; Miyata et al. (1992) J. Antibiot 45:1041-1046; Miyata et al. (1992) J. Antibiot 45:1029-1040, Fujimoto et al. (1992) FEBS Lett. 305:41-44; Oshashi et al. (1002)J. Antibiot 45:1684-1685; EP A1 0 496 452; Clozel et al. (1993) Nature 365:759-761; International Patent Application WO93/08799; Nishikibe et al. (1993) Life Sci. 52:717-724; and Benigni et al. (1993) Kidney Int. 44:440-444). Numerous sulfonamides that are endothelin peptide antagonists are also described in U.S. Pat. Nos. 5,464,853; 5,594,021; 5,591,761; 5,571,821; 5,514,691; 5,464,853; International PCT application No. 96/31492; and International PCT application No. WO 97/27979.

Further endothelin antagonists described in the following documents, incorporated herein by reference in their entirety, are exemplary of those contemplated for use in combination with the formulations provided herein: U.S. Pat. No. 5,420,123; U.S. Pat. No. 5,965,732; U.S. Pat. No. 6,080,774; U.S. Pat. No. 5,780,473; U.S. Pat. No. 5,543,521; WO 96/06095; WO 95/08550; WO 95/26716; WO 96/11914; WO 95/26360; EP 601386; EP 633259; U.S. Pat. No. 5,292,740; EP 510526; EP 526708; WO 93/25580; WO 93/23404; WO 96/04905; WO 94/21259; GB 2276383; WO 95/03044; EP 617001; WO 95/03295; GB 2275926; WO 95/08989; GB 2266890; EP 496452; WO 94/21590; WO 94/21259; GB 2277446; WO 95/13262; WO 96/12706; WO 94/24084; WO 94/25013; U.S. Pat. No. 5,571,821; WO 95/04534; WO 95/04530; WO 94/02474; WO 94/14434; WO 96/07653; WO 93/08799; WO 95/05376; WO 95/12611; DE 4341663; WO 95/15963; WO 95/15944; EP 658548; EP 555537; WO 95/05374; WO 95/05372; U.S. Pat. No. 5,389,620; EP 628569; JP 6256261; WO 94/03483; EP 552417; WO 93/21219; EP 436189; WO 96/11927; JP 6122625; JP 7330622; WO 96/23773; WO 96/33170; WO 96/15109; WO 96/33190; U.S. Pat. No. 5,541,186; WO 96/19459; WO 96/19455; EP 713875; WO 95/26360; WO 96/20177; JP 7133254; WO 96/08486; WO 96/09818; WO 96/08487; WO 96/04905; EP 733626; WO 96/22978; WO 96/08483; JP 8059635; JP 7316188; WO 95/33748; WO 96/30358; U.S. Pat. No. 5,559,105; WO 95/35107; JP 7258098; U.S. Pat. No. 5,482,960; EP 682016; GB 2295616; WO 95/26957; WO 95/33752; EP 743307; and WO 96/31492; such as the following compounds described in the recited documents: BQ-123 (Ihara, M., et al., “Biological Profiles of Highly Potent Novel Endothelin Antagonists Selective for the ET_{A} Receptor”, Life Sciences, Vol. 50(4), pp. 247-255 (1992)); PD 156707 (Reynolds, E., et al., “Pharmacological Characterization of PD 156707, an Orally Active ET_{A} Receptor Antagonist”, The Journal of Pharmacology and Experimental Therapeutics, Vol. 273(3), pp. 1410-1417 (1995)); L-754,142 (Williams, D. L., et al., “Pharmacology of L-754,142, a Highly Potent, Orally Active, Nonpeptidyl Endothelin Antagonist”, The Journal of Pharmacology and Experimental Therapeutics, Vol. 275(3), pp. 1518-1526 (1995)); SB 209670 (Ohlstein, E. H., et al., “SB 209670, a rationally designed potent nonpeptide endothelin receptor antagonist”, Proc. Natl. Acad. Sci. USA, Vol. 91, pp. 8052-8056 (1994)); SB 217242 (Ohlstein, E. H., et al., “Nonpeptide Endothelin Receptor Antagonists. VI:Pharmacological Characterization of SB 217242, A Potent and Highly Bioavailable Endothelin Receptor Antagonist”, The Journal of Pharmacology and Experimental Therapeutics, Vol. 276(2), pp. 609-615 (1996)); A-127722 (Opgenorth, T. J., et al., “Pharmacological Characterization of A-127722: An Orally Active and Highly Potent E.sub.TA—Selective Receptor Antagonist”, The Journal of Pharmacology and Experimental Therapeutics, Vol. 276(2), pp. 473-481 (1996)); TAK-044 (Masuda, Y., et al., “Receptor Binding and Antagonist Properties of a Novel Endothelin Receptor Antagonist, TAK-044 {Cyclo[D-α-Aspartyl-3-[(4-Phenylpiperazin-1-yl)Carbonyl]-L-Alanyl-L-α-Aspartyl-D-2-(2-Thienyl)Glycyl-L-Leucyl-D-Tryptophyl]Disodium Salt}, in Human Endothelin_{A} and Endothelin_{B} Receptors”, The Journal of Pharmacology and Experimental Therapeutics, Vol. 279(2), pp. 675-685 (1996)); bosentan (Ro 47-0203, Clozel, M., et al., “Pharmacological Characterization of Bosentan, A New Potent Orally Active Nonpeptide Endothelin Receptor Antagonist”, The Journal of Pharmacology and Experimental Therapeutics, Vol. 270(1), pp. 228-235 (1994)).

The formulations provided herein can also be administered in combination with other classes of compounds. Exemplary classes of compounds for combinations herein include endothelin converting enzyme (ECE) inhibitors, such as phosphoramidon; thromboxane receptor antagonists such as ifetroban; potassium channel openers; thrombin inhibitors (e.g., hirudin and the like); growth factor inhibitors such as modulators of PDGF activity; platelet activating factor (PAF) antagonists; anti-platelet agents such as GPIIb/IIIa blockers (e.g., abdximab, eptifibatide, and tirofiban). P2Y(AC) antagonists (e.g., clopidogrel, ticlopidine and CS-747), and aspirin; anticoagulants such as warfarin, low molecular weight heparins such as enoxaparin, Factor VIIa Inhibitors, and Factor Xa Inhibitors, renin inhibitors; angiotensin converting enzyme (ACE) inhibitors such as captopril, zofenopril, fosinopril, ceranapril, alacepril, enalapril, delapril, pentopril, quinapril, ramipril, lisinopril and salts of such compounds; neutral endopeptidase (NEP) inhibitors; vasopepsidase inhibitors (dual NEP-ACE inhibitors) such as omapatrilat and gemopatrilat; HMG CoA reductase Inhibitors such as pravastatin, lovastatin, atorvastatin, simvastatin, NK-104 (a.k.a. itavastatin, or nisvastatin or nisbastatin) and ZD-4522 (also known as rosuvastatin, or atavastatin or visastatin); squalene synthetase inhibitors; fibrates; bile acid sequestrants such as questran; niacin; anti-atherosclerotic agents such as ACAT inhibitors; MTP Inhibitors: calcium channel blockers such as amlodipine besylate; potassium channel activators; alpha-adrenergic agents, beta-adrenergic agents such as carvedilol and metoprolol; antiarrhythmic agents; diuretics, such as chlorothlazide, hydrochlorothiazide, flumethiazide, hydroflumethiazide, bendroflumethiazide, methylchlorothiazide, trichloromethiazide, polythiazide or benzothlazide as well as ethacrynic acid, tricrynafen, chlorthalidone, furosenilde, musolimine, bumetanide, triamterene, amiloride and spironolactone and salts of such compounds; thrombolytic agents such as tissue plasminogen activator (tPA), recombinant tPA, streptokinase, urokinase, prourokinase and anisoylated plasminogen streptokinase activator complex (APSAC); anti-diabetic agents such as biguanides (e.g. metformin), glucosidase inhibitors (e.g., acarbose), insulins, meglitinides (e.g., repaglinide), sulfonylureas (e.g., glimepiride, glyburide, and glipizide), thiozolidinediones (e.g. troglitazone, rosiglitazone and pioglitazone), and PPAR-gamma agonists; mineralocorticoid receptor antagonists such as spironolactone and eplerenone; growth hormone secretagogues; aP2 inhibitors; non-steroidal antiinflammatory drugs (NSAIDS) such as aspirin and ibuprofen; phosphodiesterase inhibitors such as PDE III inhibitors (e.g., cilostazol) and PDE V inhibitors (e.g., sildenafil, vardenafil, tadalafil); protein tyrosine kinase inhibitors; antiinflammatories; antiproliferatives such as methotrexate, FK506 (tacrolimus, Prograf), mycophenolate and mofetil; chemotherapeutic agents; immunosuppressants; anticancer agents and cytotoxic agents (e.g., alkylating agents, such as nitrogen mustards, alkyl sulfonates, nitrosoureas, ethylenimines, and triazenes): antimetabolites such as folate antagonists, purine analogues, and pyrridine analogues; antibiotics, such as anthracyclines, bleomycins, mitomycin, dactinomycin, and plicamycin; enzymes, such as L-asparaginase; farnesyl-protein transferase inhibitors; hormonal agents, such as glucocorticoids (e.g., cortisone), estrogens/antiestrogens, androgens/antiandrogens, progestins, and luteinizing hormone-releasing hormone anatagonists, octreotide acetate; microtubule-disruptor agents, such as ecteinascidins or their analogs and derivatives: microtubule-stabilizing agents such as pacitaxel (Taxol®), docetaxel (Taxotere®), and epothilones A-F or their analogs or derivatives; plant-derived products, such as vinca alkaloids, epipodophyllotoxins, taxanes; and topoisomerase inhibitors: prenyl-protein transferase inhibitors: and miscellaneous agents such as, hydroxyurea, procarbazine, mitotane, hexamethylmelamine, platinum coordination complexes such as cisplatin, satraplatin, and carboplatin); cyclosporins; steroids such as prednisone or dexamethasone; gold compounds; cytotoxic drugs such as azathiprine and cyclophosphamide: TNF-alpha inhibitors such as tenidap; anti-TNF antibodies or soluble TNF receptor such as etanercept (Enbrel) rapamycin (sirolimus or Rapamune), leflunimide (Arava); and cyclooxygenase-2 (COX-2) inhibitors such as celecoxib (Celebrex) and rofecoxib (Vioxx).

I. Article of Manufacture

Also provided are articles of manufacture, containing packaging material and a formulation of sitaxsentan sodium provided herein within the packaging material, and a label that indicates that the formulation is used for treating an endothelin-mediated disorder.

The articles of manufacture provided herein contain packaging materials. Packaging materials for use in packaging pharmaceutical products are well known to those of skill in the art. See, e.g., U.S. Pat. Nos. 5,323,907; 5,052,558; and 5,033,352. Examples of pharmaceutical packaging materials include, but are not limited to, vials, containers, syringes, bottles, and any packaging material suitable for a selected formulation and intended mode of administration and treatment.

It is understood that the foregoing detailed description and accompanying examples are merely illustrative, and are not to be taken as limitations upon the scope of the subject matter. Various changes and modifications to the disclosed embodiments will be apparent to those skilled in the art. Such changes and modifications, including without limitation those relating to the chemical structures, substituents, derivatives, intermediates, syntheses, formulations and/or methods of use provided herein, may be made without departing from the spirit and scope thereof. U.S. patents and publications referenced herein are incorporated by reference.

### EXAMPLES

### Example 1

### Preparation of 4-chloro-3-methyl-5-(2-(2-(6-methylbenzo[d][1,3]-dioxol-5-yl)acetyl)-3-thienylsulfonamido)isoxazole, sodium salt or N-(4-chloro-3-methyl-5-isoxazolyl)-2-[2-methyl-4,5-(methylenedioxy)phenylacetyl]-thiophene-3-sulfonamide, sodium salt or N-(4-chloro-3-methyl-5-isoxazolyl)-2-[3,4-(methylenedioxy)-6-methylphenylacetyl]-thiophene-3-sulfonamide, sodium salt

### A. Preparation of (4-chloro-3-methyl-5-(2-(2-(6-methylbenzo[d][1,3]dioxol-5-yl)acetyl)-3-thienylsulfonamido)isoxazole

### 1. Preparation of 5-chloromethyl-6-methylbenzo[d][1,3]dioxole

To a mixture of methylene chloride (130 L), concentrated HCl (130 L), and tetrabuylammonium bromide (1.61 Kg) was added 5-methylbenzo[d][1,3]dioxole (10 Kg) followed by the slow addition of formaldehyde (14 L, 37 wt % in water). The mixture was stirred overnight. The organic layer was separated, dried with magnesium sulfate and concentrated to an oil. Hexane (180 L) was added and the mixture heated to boiling. The hot hexane solution was decanted from a heavy oily residue and evaporated to give almost pure 5-chloromethyl-6-methylbenzo[d][1,3]dioxole as a white solid. Recrystallization from hexane (50 L) gave 5-chloromethyl-6-methylbenzo[d][1,3]dioxole (80% recovery after recrystallization).

### 2. Formation of (4-chloro-3-methyl-5-(2-(2-(6-methylbenzo[d][1,3]dioxol-5-yl)acetyl)-3-thienylsulfonamido)isoxazole

A portion of a solution of 5-chloromethyl-6-methylbenzo[d][1,3]di-oxole (16.8 g, 0.09 mol) in tetrahydrofuran (THF)(120 mL) was added to a well stirred slurry of magnesium powder, (3.3 g, 0.136 g-atom, Alfa, or Johnson-Mathey, −20+100 mesh) in THF (120 mL) at room temperature. The resulting reaction admixture was warmed to about 40-45° C. for about 2-3 min, causing the reaction to start. Once the magnesium was activated by the heating, and the reaction begun, the mixture was cooled and maintained at a temperature below about 8° C. The magnesium can be activated with dibromoethane in place of heat.

A flask containing the reaction mixture was cooled and the remaining solution of 5-chloromethlybenzo[d][1,3]dioxole added dropwise during 1.5 hours while maintaining an internal temperature below 8° C. Temperature control is important: if the Grignard is generated and kept below 8° C., no Wurtz coupling takes place. Longer times at higher temperatures promote the Wurtz coupling pathway. Wurtz coupling can be avoided by using high quality Mg and by keeping the temperature of the Grignard below about 8° C. and stirring vigorously. The reaction works fine at −20° C., so any temperature below 8° C. is acceptable at which the Grignard will form. The color of the reaction mixture turns greenish.

The reaction mixture was stirred for an additional 5 min at 0° C., while N²-methoxy-N²-methyl-3-(4-chloro-3-methyl-5-isoazolylsulfamoyl)-2-thio-phenecarboxamide (6.6 g, 0.018 mol) in anhydrous THF (90 mL) was charged into the addition funnel. The reaction mixture was degassed two times then the solution of N²-methoxy-N²-methyl-3-(4-chloro-3-methyl-5-isoxazolylsulfamoyl)-2-thio-phenecarboxamide was added at 0° C. over 5 min. TLC of the reaction mixture (Silica, 12% MeOH/CH₂Cl₂) taken immediately after the addition shows no N²-methoxy-N²-methyl-3-(4-chloro-3-methyl-5-isoxazolysulfamoyl)-2-thiophenecarboxamide.

The reaction mixture was transferred into a flask containing 1N HCl (400 mL, 0.4 mol HCl, ice-bath stirred), and the mixture stirred for 2 to 4 min, transferred into a separatory funnel and diluted with ethyl acetate (300 mL). The layers were separated after shaking. The water layer was extracted with additional ethyl acetate (150 mL) and the combined organics washed with half-brine. Following separation, THF was removed by drying the organic layer over sodium sulfate and concentrating under reduced pressure at about 39° C.

### B. Preparation of 4-chloro-3-methyl-5-(2-(2-(6-methylbenzo[d][1,3]dioxol-5yl)acetyl)-3-thienylsulfonamido)isoxazole, sodium salt

The product from part A was then re-dissolved in ethyl acetate and washed with saturated NaHCO₃ (5×50 mL) until the washings became colorless. The solution was washed with brine, dried over Na₂SO₄ and concentrated in vacuo to give a semicrystalline yellow residue. 100 mL of CH₂Cl₂ was added to the solution and the mixture stirred under nitrogen for from 5 to 10 minutes until a fine crystalline product was formed. Ether (150 mL) was added and the mixture stirred from an appropriate time (e.g., 10 min). The product was isolated by filtration, washed with a mixture of CH₂Cl₂/ether (1:2) (30 mL) then with ether (30 mL) and dried under reduced pressure. When prepared in accordance with the specific embodiments set forth above, the title product was produced in quantity of 7.3 g with a purity of around 85% (HPLC, RP, 40% acetonitrile/water, 0.1% TFA neutralized with ammonia to pH2.5, isocratic conditions, 1 mL/min).

The salt product from above was dissolved in water (600 mL) at 10° C., the solution stirred for a short period of time (e.g., 3 min) and then filtered through a layer of paper filters (e.g., 3 filters) with suction. In some cases, the large amount of impurities that are not soluble in water (10% or higher) slows down the filtration process extremely. This problem can be avoided by using a larger size filter during the filtration. Usually there is no problem with filtration if the purity of the crude salt is 90% or higher.

The greenish slightly turbid solution obtained from filtration was cooled in an ice bath and acidified to a pH of 2 using an acid such as 4N HCl. When the pH of the solution was 2, the product precipitates as a milky, non-filterable material. Slow dropwise addition of extra 4N HCl causes the product to form a fine, easily filterable precipitate. The pale yellow precipitate was filtered off, washed with water until neutral and pressed on the filter to get rid of excess of water). The obtained free acid was typically 95% pure as determined by HPLC.

The free acid form of the product was dissolved in ethyl acetate (about 100 mL), washed with brine (30 mL) to remove water. The dehydrated solution was shaken with cold saturated NaHCO₃ solution (2×30 mL), then with brine again, dried over Na₂SO₄ and concentrated in vacuo (bath temperature lower than 40° C.) to give a very bright yellow foam. After complete removal of the ethyl acetate from this product, CH₂Cl₂ (100 mL) was added and the mixture stirred for 5 to 10 min until the product became crystalline. Ether (150 mL) was added and stirring continued for 10 min longer. The formed solid was isolated by filtration, washed with a mixture of CH₂Cl₂/ether (1:2)(30 mL) then with ether (30 mL) and dried under reduced pressure. When purified in this manner, 4-chloro-3-methyl-5-(2-(2-(6-methylbenzo[d][1,3]dioxol-5-yl)acetyl)-3-thienylsulfonamido)isoxazole, sodium salt was obtained in high yield (5.7 g, 68%) with good purity (98.2% pure by HPLC). The product can also be further purified by recrystallization from EtOH/methyl t-butylether (MTBE) after the above procedure if the initial purity is sufficiently high.

### C. N-(4-Chloro-3-methyl-5-isoxazolyl)-2-[3,4-(methylenedioxy)-6-methyl]-phenylacetyl-3-thiophenesulfonamide, sodium hydrogen phosphate salt also designated 4-Chloro-3-methyl-5-(2-(2-(6-methylbenzo[d][1,3]dioxol-5-yl)acetyl)-3-thienylsulfonamido)isoxazole, sodium hydrogen phosphate salt

To a solid mixture of n-(4-chloro-3-methyl-5-isoxazolyl)-2-[3,4-(methylenedioxy)-6-methyl]phenylacetyl-3-thiophenesulfonamide (1.1492 g, 2.5263 mmol) and sodium phosphate dibasic (0.3486 g, 2.5263 mmol) was added de-ionized water (25 ml) and acetonitrile (25 ml). The resulting mixture was well shaken and warmed at 50° c. to obtain a clear solution, which was filtered. The filtrate was frozen at −78° c. and lyophilized to give the salt as a yellow powder (≈1.50 g).

Exemplary Formulations of Sitaxsentan Sodium:

The following examples provide exemplary lyophilized and tablet formulations of sitaxsentan sodium and their stability studies.

A. Lyophilized Formulations

### Example 2

### A Solution Stability Study to Determine the Effectiveness of Various Antioxidants

Stability of eight experimental antioxidant formulations was compared with the previously known formulation (See, WO 98/49162) of sitaxsentan sodium as below. The sitaxsentan sodium was present at 25 mg/mL in each of the following formulations:

I: Monothioglycerol at 10 mg/mL and disodium EDTA at 2 mg/mL in 20 mM citrate buffer at pH 6+40 mg/mL dextrose

II: Monothioglycerol at 10 mg/mL in 20 mM citrate buffer at pH 6+40 mg/mL dextrose

III: Ascorbic acid at 2 mg/mL, sodium bisulfite at 6.6 mg/mL and sodium sulfite at 2 mg/mL, 20 mM citrate pH 6+40 mg/mL dextrose

IV: Sodium sulfite at 2 mg/mL in 20 mM phosphate at pH 8+40 mg/mL dextrose

V: EDTA disodium at 2 mg/mL in 20 mM phosphate at pH 7+40 mg/mL dextrose

VI: Ascorbic acid at 2 mg/mL in 20 mM citrate at pH 6+40 mg/mL dextrose

VII: Control (see WO 98/49162): 20 mM phosphate at pH 6.8+50 mg/mL dextrose

VIII: Sodium bisulfite at 6.6 mg/mL in 20 mM citrate buffer at pH 6+40 mg/mL dextrose

IX: Sodium metabisulfite at 10 mg/mL in 20 mM citrate buffer at pH 6+40 mg/mL dextrose

These nine formulations were stored at ambient temperature and exposed to light for 48 hours. Samples were collected over time and submitted for HPLC analysis. A number of the formulations precipitated at some point however the study was continued for those samples. The studies were continued because the formulations which precipitated could be filtered and still tested by HPLC for % purity. The oxidation reaction produced a color change from yellow to orange so it was possible to visually assess the stability of the test formulations as well. In the end, the visual stability assessments correlated well with the HPLC data. The HPLC results are summarized in Tables 1 and 2.

TABLE 1
HPLC Purity Analysis of Nine Antioxidant Liquid Formulations of
sitaxsentan sodium

| Time | % Total Related Peaks From Various Formulations | | | | | | | | |
|---|---|---|---|---|---|---|---|---|---|
| Points | | | | | | | Con- | | |
| (h) | VI | III | II | I | V | IV | trol | VIII | IX |
| 0 | 0.14 | 0.23 | 0.07 | 0.14 | 0.86 | 0.39 | 3.57 | 3.01 | 11.03 |
| 2 | 0.16 | 0.21 | 0.08 | 0.14 | 1.04 | 0.57 | 4.35 | 4.01 | 6.74 |
| 4 | 0.17 | 0.24 | 0.07 | 0.14 | 1.12 | 0.62 | 4.56 | 5.26 | 8.16 |
| 24 | 0.83 | 0.32 | 0.07 | 0.36 | 4.35 | 1.01 | 11.82 | 7.43 | 7.43 |
| 48 | 0.79 | 0.42 | 0.08 | 0.49 | 6.39 | 2.23 | 15.69 | 9.64 | 9.3 |

TABLE 2
HPLC Assay of Nine Antioxidant Liquid Formulations of sitaxsentan
sodium

| Time | Assay as % of Label Claim of 25 mg/mL in Various Formulations | | | | | | | | |
|---|---|---|---|---|---|---|---|---|---|
| Points | | | | | | | Con- | | |
| (h) | VI | III | II | I | V | IV | trol | VIII | IX |
| 0 | 98.9 | 97.7 | 98.8 | 98.3 | 96.8 | 97.9 | 93.1 | 93.9 | 83.4 |
| 2 | 98.8 | 98.2 | 99.1 | 98.3 | 96.6 | 97.8 | 92.1 | 92.9 | 88.7 |
| 4 | 99.1 | 97.1 | 99.4 | 98.2 | 96.1 | 98.4 | 91.1 | 91.8 | 86.9 |
| 24 | 98.1 | 98.9 | 99.4 | 98.4 | 92.8 | 97 | 83.7 | 85.8 | 88.9 |
| 48 | 96.1 | 97.7 | 98.9 | 97.7 | 89.2 | 96.3 | 79.1 | 68.4 | 83.2 |

Table 3 contains a summary of the physical appearance of the test formulations and it can be seen that a number of them had precipitation, others experienced color changes and a few were unchanged over the course of the study.

TABLE 3
Physical Appearance of Sitaxsentan Sodium Formulations With Various
Antioxidants. Samples Stored at Ambient Temperature and Light.

| Time | Formulations | | | | | | | | |
|---|---|---|---|---|---|---|---|---|---|
| Points | V | IV | VII | VIII | IX | VI | III | II | I |
| 0 | A | A/D | A | A | A | B | A | A | A |
| 2 h | — | — | — | B | B | B | A | A | A |
| 4 h | — | D | — | B | B | B | A | A | A |
| 24 h | D | D | D | C | C | C | B | B | A |
| 48 h | D/E | D | D/E | C/F | C/F | C | B | B | A |
| Final | 6.81 | 7.97 | 6.56 | 5.78 | 5.78 | 6.48 | 6.41 | 6.53 | 6.11 |
| pH | | | | | | | | | |

A = Clear, yellow solution
B = Hazy, yellow solution
C = Hazy and/or precipitated, amber solution
D = Clear, amber solution
E = Clear, orange solution
F = Hazy and/or precipitated, orange solution

Table 4 summarizes the rank order stability of all the formulations taking into account the chemical and physical stability.

TABLE 4
Overall Rank Order Assessment of TBC Sitaxsentan Sodium
Antioxidant Formulations Considering Chemical and Physical Stability.

| | | | Physical | Overall |
|---|---|---|---|---|
| | Purity | Assay | Stability | Score |
| Formulation # | Rank | Rank | Rank | (Rank) |
| V | 6 | 6 | 3 | 15 (5) |
| IV | 5 | 4 | 2 | 11 (4) |
| Control | 9 | 7 | 3 | 19 (7) |
| VIII | 7 | 7 | 7 | 21 (8) |
| IX | 7 | 7 | 7 | 21 (8) |
| VI | 4 | 5 | 7 | 16 (6) |
| III | 2 | 1 | 5 | 8 (3) |
| II | 1 | 1 | 5 | 7 (2) |
| I | 2 | 3 | 1 | 6 (1) |

Considering all of the data, the following four formulations were carried into the lyophilization stage of the project.
I: Monothioglycerol at 10 mg/mL and disodium EDTA at 2 mg/mL in 20 mM citrate buffer at pH 6+40 mg/mL dextrose, lyophilized as lot IA
II: Monothioglycerol at 10 mg/mL in 20 mM citrate buffer at pH 6+40 mg/mL dextrose, lyophilized as lot IIA
III: Ascorbic acid at 2 mg/mL, sodium bisulfite at 6.6 mg/mL and sodium sulfite at 2 mg/mL, 20 mM citrate pH 6+40 mg/mL dextrose, lyophilized as lot IIIA
IV: Sodium sulfite at 2 mg/mL in 20 mM phosphate at pH 8+40 mg/mL dextrose, lyophilized as lot IV A

### Example 3

### Lyophilization of Samples I-IV

The four formulations above were prepared for lyophilization and were run according to the cycle summarized in Table 5.

TABLE 5
Initial Conditions for Small Scale Lyophilization of sitaxsentan sodium
Formulation with Antioxidants (Samples I-IV)

| Steps | Conditions |
|---|---|
| Step1 | Loading vials on shelf set to 5° C. |
| Step 2, Freezing | Cool shelf to −40° C. |
| Step 3, Freezing | Hold at −40° C. for 4 hours |
| Step 4, Evacuation | Evacuate chamber to a pressure of 150 mtorr |
| Step 5, Primary Drying | Heat shelf to −15° C., hold pressure |
| | at 150 mtorr |
| Step 6, Primary Drying | Hold at −15° C. and 150 mtorr for 50 hours |
| Step 7, Secondary Drying | Heat shelf to +25° C. and 50 mtorr |
| Step 8, Secondary Drying | Hold at +25° C. and 50 mtorr for a |
| | minimum of 6 hours |

Lyophilized Formula IVA exhibited a good physical cake appearance. All four formulations were submitted for moisture and HPLC analysis. All four formulations were reconstituted and their physical stability in solution was assessed. Samples were reconstituted with 10 mL of water using a needle and syringe. All samples reconstituted readily and were placed on the bench-top exposed to ambient temperature and light over a period of 48 hours (Table 6).

TABLE 6
Reconstitution Stability Study for Sitaxsentan Sodium Formulation with
Antioxidants (Formulas IA, IIA, IIIA and IVA)

| Sample | Observations/Appearance |
|---|---|
| 25 mg/mL sitaxsentan | Observations for Formula IVA |
| sodium in 20 mM | Cake dissolves with assistance of some vortex mixing. |
| Phosphate Buffer (pH | A clear yellow/golden color solution throughout the day for first 5 h. |
| 8.0 +/− 0.3), 4% | Solution was examined the following morning at 22 h and had changed back to |
| Dextrose with | initial appearance of clear yellow with no ppt after 1 week of storage at |
| 2 mg/mL Sodium | ambient temperature. |
| Sulfite | |
| 25 mg/mL sitaxsentan | Observations for Formula IIA |
| sodium in 20 mM | Cakes dissolve with hand agitation and vortex mixing. |
| Citrate Buffer (pH 6.0 +/− 0.3), | A clear light yellow solution held throughout the day for the first 5 hours. |
| 4% Dextrose | Appearance after 23 h still similar to time zero. After 28 hours of storage, the |
| with 10 mg/mL | samples began to turn a slightly hazy, light yellow solution with a white ppt |
| Monothioglycerol | forming at bottom of vials. |
| 25 mg/mL sitaxsentan | Observations for Formula IA |
| sodium in 20 mM | Cakes dissolve with hand agitation and vortex mixing. |
| Citrate Buffer (pH 6.0 +/− 0.3), | A clear, light yellow solution at time zero held for about 1 hour. |
| 4% Dextrose | At 2 h, a haze began to appear becoming very hazy within 1 h more. |
| with 10 mg/mL | At 24 h, the solution appearance was a light yellow solution with ppt settled at |
| Monothioglycerol and | the bottom of the vials. |
| 2 mg/mL of EDTA | |
| 25 mg/mL sitaxsentan | Observations for Formula IIIA |
| sodium in 20 mM | Cakes dissolve with hand agitation and vortex mixing. |
| Citrate Buffer (pH 6.0 +/− 0.3), | Samples remain clear, light yellow color throughout 24 h. |
| 4% Dextrose | No precipitation at 24 h. Solutions were clear yellow with no ppt after 1 week |
| with 2 mg/mL | of storage at ambient temp. |
| Ascorbic Acid, | |
| 6.6 mg/mL of Sodium | |
| Bisulfite, and 2 mg/mL | |
| of Sodium Sulfite | |

This data indicated that formulas IVA and IIIA were physically stable over a period of days while formulas IIA and IA precipitated within 48 hours. The HPLC data on the four lyophilized formulations is summarized in Table 7.

TABLE 7
HPLC Analysis of Various Lyophilized Formulations of sitaxsentan
sodium.

| | | Assay | % Total |
|---|---|---|---|
| Formula | Formula | (% of LC @ | Related |
| Number | Composition | 25 mg/mL) | Peaks |
| IVA | Sitaxsentan sodium at 25 mg/mL | 92.2; 92.8 | 3.57; 3.28 |
| | in 20 mM phosphate | | |
| | (pH 8) + 40 mg/mL dextrose + | | |
| | 2 mg/mL Na sulfite | | |
| IIA | Sitaxsentan sodium at 25 mg/mL | 94.8; 97.2 | 0.14; 0.14 |
| | in 20 mM citrate (pH | | |
| | 6) + 40 mg/mL dextrose + 10 mg/mL | | |
| | monothioglycerol | | |
| IA | Sitaxsentan sodium at 25 mg/mL | 97.7; 96.4 | 0.07; 0.07 |
| | in 20 mM citrate (pH | | |
| | 6) + 40 mg/mL dextrose + 10 mg/mL | | |
| | monothioglycerol + 2 mg/mL | | |
| | di-Na EDTA | | |
| IIIA | Sitaxsentan sodium at 25 mg/mL | 96.0; 95.8 | 0.08; 0.13 |
| | in 20 mM citrate (pH | | |
| | 6) + 40 mg/mL dextrose + 2 mg/mL | | |
| | ascorbic acid + 6.6 mg/mL | | |
| | Na bisulfite + 2 mg/mL | | |
| | Na sulfite | | |

From the HPLC data on the four lyophilized formulations (Table VII), it was apparent that the sodium sulfite formulation at pH 8, formula IVA, was significantly less stable than the other three formulations.

### Example 4

### Redevelopment of Formula IIA and IA

The monothioglycerol formulations were redeveloped to eliminate the precipitation while retaining the chemical stability. A number of solution formulations were set up at ambient temperature and light looking for evidence of precipitation. The following 5 formulas were examined in this study. The sitaxsentan sodium concentration was 25 mg/mL in each formula.
1: Monothioglycerol at 10 mg/mL in 20 mM citrate buffer at pH 6+40 mg/mL dextrose
2: Monothioglycerol at 10 mg/mL in 20 mM citrate buffer at pH 7+40 mg/mL dextrose
3: Monothioglycerol at 10 mg/mL in 20 mM phosphate buffer at pH 6+40 mg/mL dextrose
4: Monothioglycerol at 10 mg/mL in 20 mM phosphate buffer at pH 7+40 mg/mL dextrose
5: Monothioglycerol at 10 mg/mL in 20 mM phosphate buffer at pH 8+40 mg/mL dextrose

Formula 1 precipitated in the first 24 hours of storage and the rest of formulations were unchanged. Formula 3 precipitated approximately after 28 hours, thus indicating that the initial pH is an important factor in stabilizing the monothioglycerol formulations. The formulas at pH 7 and 8 were stable throughout longer periods of storage (>48 hours) and it seems that any of them would be acceptable to carry into lyophilization. Placebo solutions (no sitaxsentan sodium) of each formulation were monitored along with each active formula in order to learn more about the precipitation problem. None of the placebos precipitated indicating that the precipitate involves the sitaxsentan sodium.

### Example 5

### Lyophilization Studies of Formula 2 and 4

Formula 2 and 4 were lyophilized according to the cycle in Table 8

TABLE 8
Conditions for Lyophilization of 25 mg/mL Sitaxsentan Sodium in
20 mM Citrate Buffer (pH 7.0 ± 0.3)) (formulation 2A) and in
20 mM Phosphate Buffer (pH 7.0 ± 0.3), 4% Dextrose with
10 mg/mL Monothioglycerol (formulation 4A)

| Steps | Conditions |
|---|---|
| Step1 | Loading vials on shelf set to 5° C. |
| Step 2, Freezing | Cool shelf to −45° C. |
| Step 3, Freezing | Hold at −45° C. for 4 hours |
| Step 4, Evacuation | Evacuate chamber to a pressure of 150 mtorr |
| Step 5, Primary Drying | Heat shelf to −15° C. for 1 hour, hold pressure |
| | at 150 mtorr |
| Step 6, Primary Drying | Hold at −15° C. and 150 mtorr for 70 hours |
| Step 7, Secondary | Heat shelf to +25° C. for a period of |
| Drying | 80 minutes and 50 mtorr |
| Step 8, Secondary | Hold at +25° C. and 50 mtorr for a minimum |
| Drying | of 6 hours |

The physical appearance of both formulations was acceptable. The reconstitution of both formulations was good (<2 minutes). An effort was made to improve the cake appearance of the formulations by revising the lyophilization cycle. A lower freezing temperature (−45° C.) and lower primary drying temperatures (−20° C. and −25° C.) were tested and resulted in some improvement in cake appearance.

### Example 6

### Prototype Stability Study With Formula 4

Formula 4A was selected for prototype stability and was manufactured at a scale of 135 vials according to the cycle shown in Table 9. The conditions in Table 9 were selected in an effort to eliminate cake shrinkage that occurred during primary drying. Thus an extra primary drying step of −5° C. was added to the cycle.

TABLE 9
Conditions for Lyophilization of 25 mg/mL Sitaxsentan Sodium in
20 mM Phosphate Buffer (pH 7.0 ± 0.3), 4% Dextrose with
10 mg/mL Monothioglycerol for Prototype Stability

| Steps | Conditions |
|---|---|
| Step1 | Loading vials on shelf set to 5° C. |
| Step 2, Freezing | Cool shelf to −40° C. over a period of 1 hour |
| Step 3, Freezing | Hold at −40° C. for 4 hours |
| Step 4, Evacuation | Evacuate chamber to a pressure of 150 mtorr |
| Step 5, Primary Drying | Heat shelf to −15° C. over a period of |
| | 50 minutes, hold pressure at 150 mtorr |
| Step 6, Primary Drying | Hold at −15° C. and 150 mtorr for 70 hours |
| Step 7, Primary Drying | Heat shelf to −5° C. over 20 minutes, hold |
| | pressure at 150 mtorr |
| Step 8, Primary Drying | Hold at −5° C. and 150 mtorr for 4 hours |
| Step 9, Secondary | Heat shelf to +25° C. over 1 hour, and 50 mtorr |
| Drying | |
| Step 10, Secondary | Hold at +25° C. and 50 mtorr for a minimum |
| Drying | of 6 hours |

The formulations containing dextrose became difficult to reconstitute after storage and therefore was changed to corresponding formulations containing mannitol as described in Example 7.

### Example 7

### Formulations Containing Mannitol

Formulation A: Sitaxsentan sodium at 25 mg/mL, ascorbic acid at 2 mg/mL, sodium bisulfite at 6.6 mg/mL and sodium sulfite at 2 mg/mL in 20 mM citrate pH 6+mannitol at 20 mg/mL, lyophilized as shown below (Table 10):

TABLE 10
Lyophilization Conditions for formulation A

| Steps | Conditions |
|---|---|
| Step1 | Loading vials on shelf set to 5° C. |
| Step 2, Freezing | Cool shelf to −40° C. |
| Step 3, Freezing | Hold at −40° C. for 4 hours |
| Step 4, Evacuation | Evacuate chamber to a pressure of 150 mtorr |
| Step 5, Primary Drying | Heat shelf to −15° C., hold pressure at |
| | 150 mtorr |
| Step 6, Primary Drying | Hold at −15° C. and 150 mtorr for 50 hours |
| Step 7, Secondary | Heat shelf to +25° C. and 50 mtorr |
| Drying | |
| Step 8, Secondary | Hold at +25° C. and 50 mtorr for a minimum |
| Drying | of 6 hours |

Formulation B: Staxsentan sodium at 25 mg/mL and monothioglycerol at 10 mg/mL in 20 mM phosphate buffer at pH 7+mannitol at 20 mg/mL, lyophilized as shown below (Table 11):

TABLE 11
Lyophilization Conditions for formulation B

| Steps | Conditions |
|---|---|
| Step1 | Loading vials on shelf set to 5° C. |
| Step 2, Freezing | Cool shelf to −40° C. over a period of 1 hour |
| Step 3, Freezing | Hold at −40° C. for 4 hours |
| Step 4, Evacuation | Evacuate chamber to a pressure of 150 mtorr |
| Step 5, Primary Drying | Heat shelf to −15° C. over a period of 50 |
| | minutes, hold pressure at 150 mtorr |
| Step 6, Primary Drying | Hold at −15° C. and 150 mtorr for 70 hours |
| Step 7, Primary Drying | Heat shelf to −5° C. over 20 minutes, hold |
| | pressure at 150 mtorr |
| Step 8, Primary Drying | Hold at −5° C. and 150 mtorr for 4 hours |
| Step 9, Secondary | Heat shelf to +25° C. over 1 hour, and 50 mtorr |
| Drying | |
| Step 10, Secondary | Hold at +25° C. and 50 mtorr for a minimum |
| Drying | of 6 hours |

### Example 8

### A Solution Stability Study to Determine the Effect of Antioxidants: Ascorbic Acid and Monothioglycerol

Stability of three formulations containing ascorbic acid or monothioglycerol was studied. Sitaxsentan sodium was present at 25 mg/mL in each of the following formulations:

8a: Ascorbic acid at 4.0 mg/mL+20 mM citrate buffer at pH 6.8+/−0.1

8b: Ascorbic acid at 4.0 mg/mL+20 mM phosphate buffer at pH 6.8+/−0.1

8c: Monothioglycerol at 4.0 mg/mL in 20 mM phosphate buffer at pH 6.8+/−0.1

The formulations were lyophilized according to lyophilization cycle as follows: The batch was frozen to −45° C. The vacuum was started and controlled at 30 microns and then the shelf temperature was warmed to +20° C. over 10 hours and then held there until the cycle was competed based on moisture of the batch.

The lyophilized formulations were reconstituted and stored at ambient temperature and exposed to light for 48 hours. Samples were collected over time and submitted for HPLC analysis. The HPLC results are summarized in Table 8a.

| | | | | Total | Total | Total | Total |
|---|---|---|---|---|---|---|---|
| | | | | Related | Related | Related | Related |
| | | | | Products | Products | Products | Products |
| Formulation | Buffer | Excipients | mg/mL | (t = 0) | (4 h) | (24 h) | (48 h) |
| 8a | Citrate | Ascorbic Acid | 4.0 | 0.22 | 0.41 | 0.49 | 1.19 |
| 8b | Phosphate | Ascorbic Acid | 4.0 | 0.07 | 0.24 | 0.42 | 0.85 |
| 8c | Phosphate | Monothioglycerol | 4.0 | 0.21 | 0.21 | 0.25 | 0.38 |

B. Oral Tablet Formulations:

### Example 9

### Excipient Compatibility Study for Tablet Formulations

This study was designed to evaluate the effects of various diluents, binders, disintegrants, lubricants, buffering agents, and antioxidants on the stability of the drug substance. Binary mixtures of sitaxsentan sodium with various functional excipients were prepared by placing the requisite amount of drug and excipient in 20 mL glass vials and vortexing the vials for 10-15 seconds to mix the contents. These vials were opened and stored at 40° C./75% RH and tested after two and four week periods. The results in Table 12 demonstrate that, among the excipients tested, BHA, propyl gallate, and Tween 80 caused significant degradation of the drug substance. Colloidal silicon dioxide also caused significant instability to sitaxsentan sodium (86.8% drug remaining and 11.96% total related substances after four weeks at 40° C./75% RH). In addition, the following excipients promoted the degradation of the drug: dextrates, mannitol, PVP, BHT, and alpha tocopherol (greater than 1.0% total related substances and/or reduced assay after four weeks at 40° C./75% RH). These excipients were excluded from the further development studies of the tablets.

TABLE 12
Drug-Excipient Compatibility Study Results (40° C./75% RH Open Bottle)

| | | | | | Total Related | |
|---|---|---|---|---|---|---|
| | | Drug/ | Assay (%) | | Substanes (%) | |
| | | Excipient | T = 2 | T = 4 | T = 2 | T = 4 |
| Excipient Type | Excipient | (wt/wt) | Weeks | Weeks | Weeks | Weeks |
| Drug Control 1 | Sitaxsentan Sodium | N/A | 100.7 | 100.8 | 0.19 | 0.24 |
| Drug Control 2 | Sitaxsentan Sodium | N/A | 98.0 | 100.9 | 0.18 | 0.12 |
| Diluents/Fillers | Lactose Monohydrate | 1:2 | 99.3 | 100.3 | 0.28 | 0.45 |
| | (Fast-Flo) | | | | | |
| | Calcium Phosphate | 1:2 | 101.5 | 98.9 | 0.49 | 0.64 |
| | Dibasic | | | | | |
| | Microcrystalline | 1:2 | 100.0 | 99.0 | 0.63 | 0.97 |
| | Cellulose (Avicel PH- | | | | | |
| | 102) | | | | | |
| | Dextrates (Emdex) | 1:2 | 97.3 | 97.0 | 0.99 | 1.94 |
| | Mannitol | 1:2 | 96.8 | 90.0 | 2.82 | 3.97 |
| Binder/Diluent | Pregelatinized Starch | 1:2 | 98.3 | 99.6 | 0.21 | 0.31 |
| Binders | Hydroxypropyl | 1:1 | 99.8 | 99.4 | 0.28 | 0.26 |
| | Methylcellulose | | | | | |
| | (Methocel E5P) | | | | | |
| | Hydroxypropyl | 1:1 | 99.8 | 98.5 | 0.39 | 0.83 |
| | Cellulose | | | | | |
| | Polyvinylpyrrolidone | 1:1 | 97.9 | 94.7 | 1.58 | 3.80 |
| | (PVP K29/32) | | | | | |
| Disintegrants | Sodium Croscarmellose | 1:1 | 101.1 | 99.1 | 0.27 | 0.22 |
| | (Ac-Di-Sol) | | | | | |
| | Sodium Starch | 1:1 | 101.0 | 100.9 | 0.19 | 0.30 |
| | Glycolate (Explotab) | | | | | |
| Glidant/Lubricant | Magnesium Stearate | 1:1 | 100.2 | 101.3 | 0.18 | 0.15 |
| | Powdered Cellulose | 1:1 | 97.8 | 100.6 | 0.23 | 0.33 |
| | Colloidal Silicon | 1:1 | 89.9 | 86.8 | 8.99 | 11.96 |
| | Dioxide | | | | | |
| Buffering Agents | Sodium Phosphate | 2:1 | 98.9 | 99.3 | 0.21 | 0.53 |
| | Monobasic | | | | | |
| | Sodium Phosphate | 2:1 | 99.2 | 99.3 | 0.16 | 0.20 |
| | Dibasic | | | | | |
| Antioxidant | Sodium Ascorbate | 2:1 | 99.6 | 99.8 | 0.18 | 0.18 |
| | Glycine | 2:1 | 99.4 | 99.2 | 0.16 | 0.19 |
| | Sodium Metabisulfite | 2:1 | 98.2 | 99.3 | 0.11 | 0.24 |
| | Ascorbyl Palmitate | 2:1 | 100.1 | 100.5 | 0.18 | 0.20 |
| | Disodium EDTA | 2:1 | 100.3 | 100.9 | 0.23 | 0.26 |
| | BHT | 2:1 | 100.2 | 100.4 | 1.00 | 1.03 |
| | Alpha Tocopherol | 2:1 | 97.2 | 96.2 | 0.95 | 2.57 |
| | BHA | 2:1 | 77.4 | 47.6 | 18.43 | 44.95 |
| | Propyl Gallate | 2:1 | 64.0 | ND¹ | 53.91 | ND |
| Other | Tween 80 | 2:1 | 79.2 | ND | 14.63 | ND |

¹Not determined

Based on drug-excipient compatibilities, processibility, and ability to produce a tablet with satisfactory hardness and friability, lactose monohydrate and microcrystalline cellulose were chosen as diluents, hydroxypropyl methylcellulose was chosen as the binder for sitaxsentan sodium coated tablets.

### Example 10

### Effect of Coating on Tablet Formulations

Drug Stability of coated tablets containing initial prototype formulation B (Table 13) was compared to the uncoated formulation A at 40° C./75% RH.

TABLE 13
Initial Prototype Formulation B

| Component | mg per Tablet | % w/w |
|---|---|---|
| Intragranular Components | | |
| Sitaxsentan Sodium | 100.0 | 20.0 |
| Microcrystalline Cellulose (Avicel PH-102) | 175.0 | 35.0 |
| Lactose Monohydrate Fast-Flo | 84.3 | 16.9 |
| Hydroxypropyl Methylcellulose 2910 | 25.0 | 5.0 |
| Ascorbyl Palmitate | 0.5 | 0.1 |
| Sodium Starch Glycolate (Explotab) | 12.5 | 2.5 |
| Granulating Agents | | |
| Sodium Phosphate Monobasic Monohydrate | 0.6 | 0.1 |
| Granular AR | | |
| Sodium Phosphate Dibasic Anhydrous Gen | 1.1 | 0.2 |
| Disodium Edetate Dihydrate Gen AR | 0.5 | 0.1 |
| Purified Water¹ | | |
| Extragranular Components | | |
| Lactose Monohydrate Fast-Flo | 83.0 | 16.6 |
| Sodium Starch Glycoloate (Explotab) | 12.5 | 2.5 |
| Magnesium Stearate (Non-Bovine #5712) | 5.0 | 1.0 |
| Total Core Tablet Weight | 500.0 | 100.0 |

Formulation A, High Shear Granulation Process

| Component | mg per Tablet |
|---|---|
| Intragranular Components | |
| Sitaxsentan Sodium | 100.0 |
| Microcrystalline Cellulose (Avicel PH-102) | 255.8 |
| Dibasic Calcium Phosphate | 90.0 |
| Hydroxypropyl Cellulose | 20.0 |
| Sodium Phosphate Monobasic Monohydrate | 0.6 |
| Sodium Phosphate Dibasic | 1.1 |
| Sodium Starch Glycolate (Explotab) | 12.5 |
| Purified Water1 | |
| Extragranular Components | |
| Collodial Silicon Dioxide | 2.5 |
| Sodium Starch Glycolate (Explotab) | 12.5 |
| Magnesium Stearate (Non-Bovine #5712) | 5.0 |
| Total Core Tablet Weight | 500.0 |

¹In-process agent. It is removed during the process.

TABLE 14
Drug Stability of Initial Prototype Formulation as Compared to the
Original Formulation, Crushed Uncoated Tablets in Open
Bottles at 40° C./75% RH

| | T = 0 | | T = 2 Weeks | | T = 4 Weeks | |
|---|---|---|---|---|---|---|
| | Assay | TRS¹ | Assay | TRS | Assay | TRS |
| Formulation | (%) | (%) | (%) | (%) | (%) | (%) |
| Uncoated | 100.0 | 0.20 | 96.4 | 3.41 | 89.2 | 5.77 |
| formulation | | | | | | |
| Prototype coated | 97.7 | 0.06 | 92.6 | 0.70 | 91.5 | 1.46 |
| formulation | | | | | | |

¹Total related substances

As seen in Table 14, the prototype coated formulation B has improved stability as compared to the uncoated formulation A.

### Example 11

### Effect of Antioxidants

Various types of antioxidants were evaluated in the drug-excipient compatibility study (Example 10). Among the nine antioxidants evaluated, sodium ascorbate, glycine, sodium metabisulfite, ascorbyl palmitate, and disodium edetate (EDTA) were found to be compatible with the drug. The combination of ascorbyl palmitate and EDTA was chosen based on the results from the excipient compatibility studies and tablet storage stability studies. Further evaluations were conducted out to study the effects of various levels of ascorbyl palmitate (0.1%, 0.2%, and 2.0%) and EDTA (0.1% and 0.2%) on drug stability. As shown in Table 15, the formulation containing 0.2% of ascorbyl palmitate and 0.2% of EDTA is most stable over time.

TABLE 15
Effect of Antioxidant Concentration on the Stability of Sitaxsentan Sodium
100 mg Uncoated Tablets in Open Bottles at 40° C./75% RH

| Levels of | | | | | | | | | | | | |
|---|---|---|---|---|---|---|---|---|---|---|---|---|
| Antioxidants | | Tablet | T = 0 | | T = 2 Weeks | | T = 4 Weeks | | T = 8 Weeks | | T = 12 Weeks | |
| Ascorbyl | | Batch | Assay | TRS¹ | Assay | TRS | Assay | TRS | Assay | TRS | Assay | TRS |
| Palmitate | EDTA | Number | (%) | (%) | (%) | (%) | (%) | (%) | (%) | (%) | (%) | (%) |
| 0.1 | 0.1 | C | 97.7 | 0.06 | 95.1 | 0.51 | 94.2 | 1.26 | 92.5 | 2.41 | 89.8 | 3.58 |
| 0.2 | 0.2 | D | 99.1 | 0.10 | 97.8 | 0.33 | 96.3 | 0.58 | 94.5 | 1.25 | 94.2 | 1.97 |
| 2.0 | 0.1 | E | 98.8 | 0.12 | 95.6 | 1.15 | 96.5 | 1.52 | 93.3 | 2.35 | 93.1 | 3.29 |

¹Total related substances

Formulation C, D and E with Various Levels of Antioxidants
(mg per Tablet)

| | | | |
|---|---|---|---|
| Component | C | D | E |

Intragranular Components

| | | | |
|---|---|---|---|
| Sitaxsentan Sodium | 100.0 | 100.0 | 100.0 |
| Microcrystalline Cellulose (Avicel | 175.0 | 175.0 | 175.0 |
| PH-102) | | | |
| Lactose Monohydrate Fast-Flo | 84.3 | 84.3 | 84.3 |
| Hydroxypropyl Methylcellulose | 25.0 | 25.0 | 25.0 |
| 2910 | | | |
| Ascorbyl Palmitate | 0.5 | 1.0 | 10.0 |
| Sodium Starch Glycolate | 12.5 | 12.5 | 12.5 |
| (Explotab) | | | |
| Sodium Phosphate Monobasic | 0.6 | 0.6 | 0.6 |
| Monohydrate Granular AR | | | |
| Sodium Phosphate Dibasic | 1.1 | 1.1 | 1.1 |
| Anhydrous Gen | | | |
| Disodium Edetate Dihydrate Gen | 0.5 | 1.0 | 0.5 |
| AR | | | |
| Purified Water¹ | | | |

Extragranular Components

| | | | |
|---|---|---|---|
| Lactose Monohydrate Fast-Flo | 83.0 | 82.0 | 73.5 |
| Sodium Starch Glycolate | 12.5 | 12.5 | 12.5 |
| (Explotab) | | | |
| Magnesium Stearate (Non-Bovine | 5.0 | 5.0 | 5.0 |
| #5712) | | | |
| Total Core Tablet Weight | 500 | 500 | 500 |

¹In-process agent. It is removed during the process.

Test Method: HPLC with a Diode Array detector (264 nm and 240 nm). Column: phenomenex Luna C18 (2) 4.6 mm×150 mm, 5 micron particles. Mobil Phases: 50 mN H₃PO₄ at pH 3.5 and Methanol.

### Example 12

### Effect of Buffers

A buffer agent mixture is used to improve drug stability in the tablets. A sodium phosphate monobasic (0.1% wt/wt) and sodium phosphate dibasic (0.2% wt/wt) buffer mixture (buffer pH 6.8) was found to improve the drug stability relative to the control tablet without the buffer salts (Table 16). Therefore, the buffer salts mixture was used in the formulation to control the microenvironment of the drug substance during the granulation process and in the resulting tablet.

TABLE 16
Effect of Buffering Salts on the Stability of Sitaxsentan Sodium 100 mg
Uncoated Tablets in Open Bottles at 40° C./75% RH

| | Tablet | T = 0 | | T = 2 Weeks | | T = 1 Month | | T = 2 Months | |
|---|---|---|---|---|---|---|---|---|---|
| | Batch | Assay | TRS¹ | Assay | TRS | Assay | TRS | Assay | TRS |
| Buffer Agent | Number | (%) | (%) | (%) | (%) | (%) | (%) | (%) | (%) |
| 0.1% Sodium | F | 99.3 | 0.39 | 97.9 | 0.49 | 96.4 | 1.25 | 94.0 | 2.55 |
| Phosphate Monobasic | | | | | | | | | |
| and 0.2% Sodium | | | | | | | | | |
| Phosphate Dibasic | | | | | | | | | |
| (Uncoated Tablet) | | | | | | | | | |
| No Buffer Control | G | 98.3 | 0.42 | 95.3 | 1.10 | 92.6 | 2.40 | 88.4 | 4.08 |
| (4% | | | | | | | | | |
| Sepifilm ®/Sepisperse | | | | | | | | | |
| Coating) | | | | | | | | | |

¹Total related substances

TABLE 17
Formulation F and G With and Without Buffer
Agents

mg per Tablets

| Component | F | G |
|---|---|---|
| Intragranular Components | | |
| Sitaxsentan Sodium | 100.0 | 100.0 |
| Microcrystalline Cellulose (Avicel PH- | 175.0 | 175.0 |
| 102) | | |
| Lactose Monohydrate Fast-Flo | 84.3 | 84.3 |
| Hydroxypropyl Methylcellulose 2910 | 25.0 | 25.0 |
| Ascorbyl Palmitate | 0.5 | 10.0 |
| Sodium Starch Glycolate (Explotab) | 12.5 | 12.5 |
| Sodium Phosphate Monobasic | 0.6 | 0.0 |
| Monohydrate Granular AR | | |
| Sodium Phosphate Dibasic Anhydrous | 1.1 | 0.0 |
| Gen | | |
| Disodium Edetate Dihydrate Gen AR | 0.5 | 0.5 |
| Purified Water1 | | |
| Extragranular Components | | |
| Lactose Monohydrate Fast-Flo | 83.0 | 84.7 |
| Sodium Starch Glycolate (Explotab) | 12.5 | 12.5 |
| Magnesium Stearate (Non-Bovine | 5.0 | 5.0 |
| #5712) | | |
| Total Core Tablet Weight | 500 | 500 |

¹In-process agent. It is removed during the process.

### Example 13

### Effect of Coating

Four types of coatings were initially evaluated, Sepifilm® LP014/Sepisperse Dry 3202 Yellow, Blue Opadry, Eudragit EPO and Opadry AMB. The main objective was to identify a coating that would serve as a moisture barrier to further hinder oxidation of sitaxsentan sodium. Among the four types of coating materials evaluated, Sepifilm® LP014/Sepisperse Dry 3202 Yellow (Sepifilm®/Sepisperse) (3/2 wt/wt) at a 4% tablet weight gain and Blue Opadry at 3% tablet weight gain both produced a uniform smooth coating. Sepifilm® LP014/Sepisperse Dry 3202 Yellow (Sepifilm®/Sepisperse) (3/2 wt/wt) at a 4% tablet weight gain was selected because of its good processibility.

TABLE 18
Effect of Coating on the Stability of Sitaxsentan Sodium 100 mg Tablets
in Open Bottles at 40° C./75% RH (Test Method described Example 11)

| | T = 0 | | T = 2 Weeks | | T = 4 Weeks | | T = 8 Weeks | | T = 12 Weeks | |
|---|---|---|---|---|---|---|---|---|---|---|
| | Assay | TRS¹ | Assay | TRS | Assay | TRS | Assay | TRS | Assay | TRS |
| Formulation | (%) | (%) | (%) | (%) | (%) | (%) | (%) | (%) | (%) | (%) |
| C | 97.7 | 0.06 | 95.1 | 0.51 | 94.2 | 1.26 | 92.5 | 2.41 | 89.8 | 3.58 |
| Uncoated | | | | | | | | | | |
| H | 97.9 | 0.11 | 96.3 | 0.57 | 93.7 | 1.51 | 91.9 | 2.70 | 90.4 | 4.26 |
| 4% Opadry AMB | | | | | | | | | | |
| I | 95.6 | 0.11 | 94.7 | 0.62 | 92.3 | 1.76 | 90.6 | 3.30 | 87.8 | 4.77 |
| 4% Eudragit EPO | | | | | | | | | | |
| J | 97.9 | 0.10 | 95.1 | 0.43 | 94.6 | 1.22 | 91.9 | 2.39 | 89.5 | 3.46 |
| 4% | | | | | | | | | | |
| Sepifilm ®/Sepisperse | | | | | | | | | | |

¹Total related substances

Formulation have the same tablet core as tablet C. Coating are different as described in Table 18.

### Example 14

### Sitaxsentan 100 mg Coated Tablets

The tablets were manufactured on a one kg scale. The granulating solution was prepared by dissolving sodium phosphate, mono- and di-basic, and disodium EDTA in purified water. Ascorbyl palmitate was added to the sitaxsentan sodium drug substance and blended in a bag by hand for approximately 30 seconds. Approximately half of the microcrystalline cellulose was added to the bag and blended for an additional 30 seconds. The mixture was screened through a screen. The remaining intragranular components (i.e., remaining microcrystalline cellulose, lactose, HPMC, sodium starch glycolate) were screened through a screen and added to the mixture. The powders were then charged into a heated Glatt GPCG-1. The granulating solution was applied to the intragranular powders. Additional water was sprayed, if necessary, to achieve a visually desirable granulation. After that, the granulation was dried until an LOD of less than 2% was achieved. The dried granulation was milled through a Fitzmill with a 0.0024-sized screen. Extragranular components were screened and blended with the milled granulation in an 8-qt. V-blender for five minutes. Magnesium stearate was screened then blended with the mixture for three minutes. The final blends were compressed on a tablet press to 500 mg core tablets using 0.2900″×0.6550″ modified oval tooling.

Coating suspension was prepared by adding Sepifilm® LP014 and Sepisperse Dry 3202 (Yellow) to water with mixing. Mixing continued until a homogenous suspension is formed. The tablets were coated using a Compu-lab coater with a 19″ coating pan.

TABLE 19
Sitaxsentan Sodium 100 mg Clinical Tablet Formulation

| Component | mg/tablet | % w/w |
|---|---|---|
| Sitaxsentan sodium | 100.0 | 20.0 |
| Microcrystalline Cellulose (Avicel PH 102) | 175.0 | 35.0 |
| Lactose Monohydrate Fast Flo (intragranular) | 84.3 | 16.9 |
| Lactose Monohydrate Fast Flo (extragranular) | 82.0 | 16.4 |
| Hydroxypropyl Cellulose E-5P | 25.0 | 5.0 |
| Ascorbyl Palmitate | 1.0 | 0.2 |
| EDTA, Disodium | 1.0 | 0.2 |
| Sodium Phosphate, Monobasic Monohydrate | 0.6 | 0.1 |
| Sodium Phosphate, Dibasic Anhydrous | 1.1 | 0.2 |
| Sodium Starch Glycolate (intragranular) | 12.5 | 2.5 |
| Sodium Starch Glycolate (extragranular) | 12.5 | 2.5 |
| Magnesium Stearate, Non-Bovine | 5.0 | 1.0 |
| Purified Water, USP | 192.5 | — |
| Total Core Tablet Weight | 500.0 | 100.0 |
| Sepisperse Dry 3202 (Yellow) | 8.0 | 1.6 |
| Sepifilm ® LP 014 | 12.0 | 2.4 |
| Total Coated Tablet Weight | 520.0 | 104.0 |

### Example 15

### Comparison between the uncoated tablet core and the coated tablet

Comparison between the uncoated tablet core and the coated tablet, prepared by the method of Example 14, was conducted to determine the effect of the Sepifilm®/Sepisperse moisture barrier.

TABLE 20
Stability Results of the Formulation for Sitaxsentan Sodium 100 mg
Uncoated Tablet Core

| | T = 0 | | T = 1 Month | | T = 3 Months | | T = 6 Months | |
|---|---|---|---|---|---|---|---|---|
| Storage | Assay | TRS¹ | Assay | TRS | Assay | TRS | Assay | TRS |
| Conditions | (%) | (%) | (%) | (%) | (%) | (%) | (%) | (%) |
| 25° C./60% RH² | 100.0 | 0.06 | 101.9 | 0.00 | 98.9 | 0.00 | 99.3 | 0.27 |
| 40° C./75% RH | 100.0 | 0.06 | 97.7 | 0.00 | 98.0 | 0.29 | 95.5 | 1.66 |

¹Total related substances
²Relative humidity

TABLE 21
Stability Results of the Formulation for Sitaxsentan Sodium 100 mg
Coated Tablet Core

| | | | T = 1 | | T = 2 | | T = 3 | | T = 6 | |
|---|---|---|---|---|---|---|---|---|---|---|
| | T = 0 | | Month | | Months | | Months | | Months | |
| Storage | Assay | TRS1 | Assay | TRS | Assay | TRS | Assay | TRS | Assay | TRS |
| Conditions | (%) | (%) | (%) | (%) | (%) | (%) | (%) | (%) | (%) | (%) |
| 25° C./60% RH² | 99.5 | 0.00 | 98.5 | 0.00 | 99.5 | 0.00 | 98.6 | 0.00 | 98.2 | 0.23 |
| 40° C./75% RH | 99.5 | 0.00 | 98.3 | 0.05 | 97.8 | 0.06 | 98.4 | 0.32 | 98.2 | 1.02 |

¹Total related substances
²Relative humidity

As seen from the results in Tables 20 and 21 above, the Sepifilm®/Sepisperse coating provides additional protection for the drug substance in the tablet.

All of the references cited herein are incorporated by reference in their entirety. While the invention has been described with respect to the particular embodiments, it will be apparent to those skilled in the art that various changes and modifications may be made without departing from the spirit and scope of the invention as recited by the appended claims.

The embodiments described above are intended to be merely exemplary, and those skilled in the art will recognize, or will be able to ascertain using no more than routine experimentation, numerous equivalents of specific compounds, materials, and procedures. All such equivalents are considered to be within the scope of the invention and are encompassed by the appended claims.

What is claimed is:

1. A lyophilized powder comprising sitaxsentan sodium, an antioxidant, a buffer and a bulking agent.

2. The lyophilized powder of claim 1, wherein the sitaxsentan sodium is present in an amount from about 20% to about 50% by total weight of the lyophilized powder.

3. The lyophilized powder of claim 1, wherein the amount of sitaxsentan sodium is about 41% by total weight of the lyophilized powder.

4. The lyophilized powder of claim 1, wherein the antioxidant is sodium sulfite, sodium bisulfite, sodium metasulfite, monothioglycerol, ascorbic acid or a combination thereof.

5. The lyophilized powder of claim 1, wherein the antioxidant is monothioglycerol.

6. The lyophilized powder of claim 1, wherein the antioxidant is a combination of ascorbic acid, sodium sulfite and sodium bisulfite.

7. The lyophilized powder of claim 5, wherein the monothioglycerol in the lyophilized powder is present in an amount ranging from about 10% to about 30% by total weight of the lyophilized powder.

8. The lyophilized powder of claims 4, wherein the ascorbic acid is present in an amount from about 1% to about 5% by total weight of the lyophilized powder.

9. The lyophilized powder of claim 8, wherein the amount of ascorbic acid is about 3.3% by total weight of the lyophilized powder.

10. The lyophilized powder of claims 4, wherein the sodium sulfite is present in an amount from about 1% to about 5% by total weight of the lyophilized powder.

11. The lyophilized powder of claims 10, wherein the amount of sodium sulfite is about 3.3% by total weight of the lyophilized powder.

12. The lyophilized powder of claim 4, wherein the sodium bisulfite is present in an amount from about 5% to about 20% by total weight of the lyophilized powder.

13. The lyophilized powder of claims 12, wherein the amount of sodium bisulfite is about 10.8% by total weight of the lyophilized powder.

14. The lyophilized powder of claim 4, wherein the amount of ascorbic acid is about 2 mg, sodium sulfite is about 3.3% and sodium bisulfite is about 10.8% by total weight of the lyophilized powder.

15. The lyophilized powder of claim 1, wherein the buffer is a phosphate or citrate buffer.

16. The lyophilized powder of claim 1, wherein the buffer is sodium citrate dihydrate.

17. The lyophilized powder of claim 16, wherein the amount of sodium citrate dihydrate is about 8.8% by the total weight of the lyophilized powder.

18. The lyophilized powder of claim 1, wherein the bulking agent is selected from a sugar, a polyalcohol, an amino acid, a polymer and a polysaccharide.

19. The lyophilized powder of claim 1, wherein the bulking agent is sorbitol, mannitol or dextrose.

20. The lyophilized powder of claim 19, wherein the bulking agent is dextrose.

21. The lyophilized powder of claim 20, wherein the dextrose is present in an amount ranging from about 15% to about 50% by total weight of the lyophilized powder.

22. The lyophilized powder of claim 18, wherein the sugar is mannitol.

23. The lyophilized powder of claim 22, wherein the mannitol is present in an amount ranging from about 15% to about 45% by total weight of the lyophilized powder.

24. The lyophilized powder of claim 23, wherein the amount of mannitol is about 32.8% by total weight of the lyophilized powder.

25. The lyophilized powder of claim 1 comprising about 41% of sitaxsentan sodium, about 3.3% ascorbic acid, about 3.3% sodium sulfite and about 10.8% sodium bisulfite, about 8.8% sodium citrate dihydrate and about 32.8% mannitol.

26. The lyophilized powder of claim 1 comprising about 33% of sitaxsentan sodium, about 5.3% ascorbic acid, about 7.6% sodium citrate dihydrate, about 53% D-mannitol and about 0.13% citric acid monohydrate by total weight of the lyophilized powder.

27. The lyophilized powder of claim 1, comprising about 34% of sitaxsentan sodium, about 5.5% ascorbic acid, about 3.7% sodium phosphate dibasic heptahydrate, about 55% D-mannitol and about 1.9% sodium phosphate monobasic monohydrate by total weight of the lyophilized powder.

28. A reconstituted formulation of sitaxsentan sodium, wherein the reconstituted solution comprises the lyophilized powder of claim 1.

29. The reconstituted formulation of claim 28, wherein the formulation has a pH from about 5 to about 10.

30. The reconstituted formulation of claim 28, wherein the formulation has a pH of about 6.

31. The reconstituted formulation of claim 28, wherein the formulation has a pH of about 6.8.

32. An oral tablet comprising sitaxsentan sodium, an antioxidant, a binding agent, a diluent, a buffer and a moisture resistant coating.

33. The oral tablet of claim 32, wherein the sitaxsentan sodium is present in an amount ranging from about 5% to about 40% of the total weight of the tablet.

34. The oral tablet of claims 33, wherein the amount of sitaxsentan sodium is from about 15% to about 25% of the total weight of the tablet.

35. The oral tablet of any claims 33, wherein the amount of sitaxsentan sodium is from about 20% of the total weight of the tablet.

36. The oral tablet of claim 33, wherein the amount of sitaxsentan sodium is about 100 mg.

37. The oral tablet of claim 32, wherein the antioxidant is a combination of ascorbyl palmitate and EDTA, disodium.

38. The oral tablet of claim 37, wherein the ascorbyl palmitate is present in an amount ranging from about 0.05% to about 3% of the total weight of the tablet.

39. The oral tablet of claim 38, wherein the amount of ascorbyl palmitate is about 0.2% of the total weight of the tablet.

40. The oral tablet of claim 38, wherein the ascorbyl palmitate is present in an amount ranging from about 0.1 mg to about 5 mg.

41. The oral tablet of claim 38, wherein the amount of ascorbyl palmitate is about 1 mg.

42. The oral tablet of claim 37, wherein the EDTA, disodium is present in an amount ranging from about 0.05% to about 3% of the total weight of the tablet.

43. The oral tablet of claim 42, wherein the amount of EDTA, disodium is about 0.2% of the total weight of the tablet.

44. The oral tablet of claim 37, wherein the EDTA, disodium is present in an amount ranging from about 0.1 mg to about 5 mg.

45. The oral tablet of claim 44, wherein the amount of EDTA, disodium is about 1 mg.

46. The oral tablet of claim 32, wherein the diluent comprises a combination of lactose monohydrate fast flo as an intragranular component and lactose monohydrate fast flo as an extragranular component.

47. The oral tablet of claim 46, wherein the intragranula lactose monohydrate fast flo is in an amount from about 5% to about 30% and the extragranular lactose monohydrate fast flo is in an amount from about 5% to about 30% of the total weight of the tablet.

48. The oral tablet of claim 46, wherein the amount of intragranular lactose monohydrate fast flo is about 16.9% and the amount of extragranular lactose monohydrate fast flo is about 16.4% of the total weight of the tablet.

49. The oral tablet of claim 46, wherein the amount of intragranular lactose monohydrate fast flo is about 84.3 mg and the amount of extragranular lactose monohydrate fast flo is about 82 mg.

50. The oral tablet of claim 32, further comprising microcrystalline cellulose in an amount from about 10% to about 50% of the total weight of the tablet.

51. The oral tablet of claim 50, wherein the amount of microcrystalline cellulose is about 35% of the total weight of the tablet.

52. The oral tablet of claim 50, wherein the amount of microcrystalline cellulose is from about 130 mg to about 300 mg.

53. The oral tablet of claim 52, wherein the amount of microcrystalline cellulose is about 175 mg.

54. The oral tablet of claim 32, wherein the binding agent is hydroxypropyl methylcellulose (E-5P).

55. The oral tablet of claim 54, wherein hydroxypropyl methylcellulose (E-5P) is in an amount raging from about 10% to about 50% of the total weight of the tablet.

56. The oral tablet of claim 55, wherein the amount of hydroxypropyl methylcellulose (E-5P) is about 5% of the total weight of the tablet.

57. The oral tablet of claim 55, wherein the amount of hydroxypropyl methylcellulose (E-5P) is about 25 mg.

58. The oral tablet of claim 32, wherein the moisture resistant coating comprises hydroxypropylmethylcellulose from about 1% to about 6% of the total weight of the tablet.

59. The oral tablet of claim 58, wherein the coating comprises hydroxypropylmethylcellulose from about 8 mg to about 12 mg per tablet.

60. The oral tablet of claim 32, wherein the tablet comprises sitaxsentan sodium; microcrystalline cellulose; lactose monohydrate fast flo; hydroxypropyl methylcellulose E-5P; ascorbyl palmitate; disodium EDTA; sodium phosphate monobasic, monohydrate; sodium phosphate dibasic, anhydrous; sodium starch glycoloate; magnesium stearate and a moisture resistant coating of hydroxypropylmethylcellulose.

61. The oral tablet of claim 32, wherein the tablet comprises about 20% sitaxsentan sodium; about 35% microcrystalline cellulose; about 16.9% intragranular lactose monohydrate fast flo; about extragranular 16.4% lactose monohydrate fast flo; about 5.0% hydroxypropyl methylcellulose E-5P; about 0.2% ascorbyl palmitate; about 0.2% disodium EDTA; about 0.1% sodium phosphate monobasic, monohydrate; about 0.2% sodium phosphate dibasic, anhydrous; about 2.5% extragranular sodium starch glycoloate; about 2.5% intragranular sodium starch glycoloate; about 1% magnesium stearate and a moisture resistant coating of hydroxypropylmethylcellulose at about 2.4%/1.6% weight gain.

62. The oral tablet of claim 32, wherein the tablet comprises about 100 mg sitaxsentan sodium; about 1.0 mg ascorbyl palmitate; about 1.0 mg disodium edetate, EDTA; about 25 mg hydroxypropyl methylcellulose E-5P; about 84.3 intragranular lactose monohydrate fast flo; about 82 mg extragranular lactose monohydrate fast flo; about 175 mg microcrystalline cellulose; about 0.6 mg sodium phosphate monobasic, monohydrate; about 1.1 mg sodium phosphate dibasic, anhydrous; about 12.5 mg extragranula sodium starch glycoloate, about 12.5 mg intragranular sodium starch glycoloate; about 5 mg magnesium stearate and a moisture resistant coating of hydroxypropylmethylcellulose at about 20 mg.

63. A combination, comprising the formulation of claim 1 and a sterile vessel containing a single dosage or multiple dosage amount thereof.

64. The combination of claim 63, wherein the vessel is an ampoule, vial or syringe.

65. A pharmaceutical composition formulated for single dosage or multiple dosage administration prepared by mixing a single dosage of the formulation of claim 1 with an aqueous medium.

66. A method for the treatment of an endothelin-mediated disease, comprising administering an effective amount of the formulation of claim 1.

67. The method of claim 66, wherein the disease is selected from the group consisting of hypertension, cardiovascular disease, asthma, pulmonary hypertension, inflammatory diseases, opthalmologic disease, menstrual disorders, obstetric conditions, wounds, gastroenteric disease, renal failure, immunosuppressant-mediated renal vasoconstriction, erythropoietin-mediated vasoconstriction endotoxin shock, anaphylactic shock and hemorrhagic shock.

68. An article of manufacture comprising packaging material and a formulation of claim 1, contained within the packaging material, wherein the packaging material includes a label that indicates that the formulation is used for treating an endothelin mediated disorder.

69. A method for the treatment of an endothelin-mediated disease, comprising administering an effective amount of the formulation of claim 32.

70. The method of claim 69, wherein the disease is selected from the group consisting of hypertension, cardiovascular disease, asthma, pulmonary hypertension, inflammatory diseases, opthalmologic disease, menstrual disorders, obstetric conditions, wounds, gastroenteric disease, renal failure, immunosuppressant-mediated renal vasoconstriction, erythropoietin-mediated vasoconstriction endotoxin shock, anaphylactic shock and hemorrhagic shock.

71. An article of manufacture comprising packaging material and a formulation of claim 32, contained within the packaging material, wherein the packaging material includes a label that indicates that the formulation is used for treating an endothelin mediated disorder.

69. A process for preparing a lyophilized powder, comprising:
mixing sitaxsentan sodium with a solution comprising an antioxidant, a buffer and a sugar to produce a solution thereof; and
lyophilizing the solution to produce a powder.

70. An oral tablet comprising sitaxsentan sodium and a buffer.

71. An oral tablet comprising sitaxsentan sodium and a moisture barrier coating.

72. An oral tablet comprising sitaxsentan sodium and an antioxidant.
